(19) United States
(12) Patent Application Publication
(10) Pub. No.: US 2006/0258630 A1
(43) Pub. Date: Nov. 16, 2006

(54) Method for the prevention and treatment of uveitis

(75) Inventor: Luciano Adorini, Milan (IT)

Correspondence Address: EDWARDS & ANGELL, LLP, P.O. BOX 55874, BOSTON, MA, 02205, US

(73) Assignee: BioXell S.p.A., Milan (IT)

(21) Appl. No.: 11/386,494

(22) Filed: Mar. 21, 2006

Related U.S. Application Data
(63) Continuation-in-part of application No. PCT/US04/31412, filed on Sep. 24, 2004.
(60) Provisional application No. 60/505,735, filed on Sep. 24, 2003.
(60) Provisional application No. 60/718,766, filed on Sep. 19, 2005.

(30) Foreign Application Priority Data
Sep. 24, 2003 (GB) 0322395.5
Mar. 1, 2004 (GB) 0404567.0

(51) Int. Cl. A61K 31/59

(52) U.S. Cl. 514/167

(57) ABSTRACT

The invention provides 1,3-di-acylated vitamin D₃ analogs of cholecalciferol, substituted at carbon-20 with methyl or cyclopropyl wherein carbon-16 is a single or double bond, and carbon-23 is a single, double, or triple bond. Various alkyl or haloalkyl substitutions are incorporated at carbon-25. The invention provides pharmaceutically acceptable esters, salts, and prodrugs thereof. Methods for using the compounds for the prevention and treatment of uveitis are also disclosed.

### RELATED APPLICATIONS

This application is a continuation-in-part of international application PCT/US04/31412, filed 24 Sep. 2004, which designated the United States and was published in English as international publication W02005/030222 on 7 Apr. 2005, which claims priority to: U.S. provisional application Ser. No. 60/505,735, filed 24 Sep. 2003; GB0322395.5, filed 24 Sep. 2003; and GB0404567.0, filed 1 Mar. 2004. This application also claims the benefit of U.S. provisional application Ser. No. 60/718,766, filed 19 Sep. 2005. Each of the aforementioned applications is incorporated herein in its entirety by this reference.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorus homeostasis.

Studies involving the metabolism of vitamin D₃ were initiated with the discovery and chemical characterization of the plasma metabolite, 25-hydroxyvitamin D₃ [25(OH) D₃] (Blunt, J. W. et al. (1968) Biochemistry 6:3317-3322) and the hormonally active form, 1-alpha,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1-alpha,25(OH)₂D₃ in a carefully regulated fashion (Fraser, D. R. and Kodicek, E (1970) Nature 288:764-766; Wong, R. G. et al. (1972) J. Clin. Invest. 51:1287-1291), and the discovery of a nuclear receptor for 1-alpha,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427432; Ohyama, Y and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1-alpha,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1-alpha,25(OH)₂D₃ to generate the requisite specific biological responses for this secosteroid hormone (Pike, J. W. (1991) Annu. Rev. Nutr. 11:189-216). To date, there is evidence that nuclear receptors for 1-alpha,25(OH)₂D₃ (VD₃R) exist in more than 30 tissues and cancer cell lines (Reichel, H. and Norman, A. W. (1989) Annu. Rev. Med. 40:71-78), including the normal eye (Johnson J A et al. Curr Eye Res. February 1995; 14(2): 101-8).

Vitamin D₃ and its hormonally active forms are well-known regulators of calcium and phosphorus homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1-alpha,25(OH)₂ D₃ has been suggested by the combined presence of enzymes capable of oxidizing vitamin D₃ into its active forms, e.g., 25-OHD-1-alpha-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogues of these compounds. A large number of these analogues involve structural modifications in the A ring, B ring, C/D rings, and, primarily, the side chain (Bouillon, R. et al. (1995) Endocrine Reviews 16(2):201-204). Although a vast majority of the vitamin D₃ analogues developed to date involve structural modifications in the side chain, a few studies have reported the biological profile of A-ring diastereomers (Norman, A. W. et al. (1993) J. Biol. Chem. 268 (27): 20022-20030). Furthermore, biological esterification of steroids has been studied (Hochberg, R. B., (1998) Endocr. Rev. 19(3): 331-348), and esters of vitamin D₃ are known (WO 97/11053).

Moreover, despite much effort in developing synthetic analogues, clinical applications of vitamin D and its structural analogues have been limited by the undesired side effects elicited by these compounds after administration to a subject for known indications/applications of vitamin D compounds.

Uveitis, a condition comprising inflammation of the eye including the iris, ciliary body, and choroid, actually comprises a large group of diverse diseases affecting not only the uvea but also the retina, optic nerve and vitreous. According to the International Uveitis Study Group, there are several classifications of uveitis: anterior, intermediate, posterior and panuveitis (total). Inflammation may be induced by trauma or toxic or infectious agents, but in most cases the mechanisms seem to be autoimmune in nature. Symptoms may be acute, sub-acute, chronic (greater than 3 months duration) and recurrent. The etiology is unknown in the majority of cases of endogenous uveitis. Uveitis is a major cause of severe visual impairment. Although the number of patients blinded from uveitis is unknown, it has been estimated that uveitis accounts for 10-15% of all cases of total blindness in the USA. A variety of conditions can be described as posterior uveitis: focal, multifocal or diffuse choroiditis, chorioretinitis, retinochoroiditis, uveoretinitis or neurouveitis. The condition is usually painless but is characterised by the presence of floaters, vision impairment (sudden or gradual) such as blurring of vision, etc., and vision loss. Posterior uveitis may have several etiologies, and manifests itself in complex and sometimes misleading clinical conditions. There is growing evidence both in experimental models and clinically that endogenous posterior uveoretinitis is often characterised by an exaggerated immune response which causes tissue destruction. When no apparent infectious or neoplastic aetiology is found, treatment can be directed towards dampening the resulting inflammatory cascade and hopefully reducing tissue damage.

The mainstay of treatment is systemic corticosteroid and often this is given in combination with immunosuppressive agents, such as cyclosporin A or azathioprine. High dose steroids are often required to control the disease and in addition to the disadvantages of the required longterm use and resistance in some patients, potentially serious side effects are often present. In young people a common side effect is weight gain, particularly around the face, which can be cosmetically unacceptable. Another important side effect of corticosteroids, particularly with reference to the eye is glaucoma resulting from increased intraocular pressure.

Furthermore, systemic treatment with corticosteroids is often inefficient in treating the macular edema that can complicate posterior uveitis. This inefficiency can be partially overcome by intravitreal administration of such drugs, though this route of administration has the obvious drawback of patient comfort and, despite the localisation of the administration, poor drug penetration into ocular tissues often occurs.

Therefore a strong need exists for more selective and specific uveitis immunotherapy to be developed that is amenable to systemic administration and which is free of the well recognised disadvantages of the current treatments.

### BRIEF DESCRIPTION OF THE DRAWINGS

The present invention is further described below with reference to the following non-limiting examples and with reference to the following figures, in which:

FIG. 1 shows the the experimental procedure used to treat experimental autoimmune uveoretinitis(EAU) with Compound A (1,3-Di-O-acetyl-1,25-dihydroxy-20-cyclopropyl-cholecalciferol.

FIG. 2 shows the EAU disease score (quantitated between 0 and 4) at day 21.

FIG. 3 shows the reduced antigen-specific delayed type hypersensitivity (DTH) responses to IRBP in mice.

FIG. 4 shows the in vitro assay of primed lymph node cells (LN) used to indicated that calcitriol and Compound A both appeared to be potent on T cell polarization.

FIG. 5 shows that Ag driven chemokine release, such as MIP-1a, Rantes and TARC, is inhibited by Vitamin D3 derivatives.

### SUMMARY OF THE INVENTION

The present invention provides a new method of treating uveitis with a view to mitigating or alleviating the aforementioned disadvantages. The method is based on the use of calcitriol analogs, collectively “vitamin D compounds”. As described in the Examples herein, analogs of calcitriol can prevent experimental autoimmune uveoretinitis (EAU), an autoimmune disease mediated by Th1-type uveitogenic CD4+ T cells that serves as a model for human posterior uveitis.

Thus, in one aspect, the invention provides a method of prevention or treatment of uveitis using a vitamin D compound of the invention.

In another aspect, the invention provides a method for preventing or treating uveitis in a subject, comprising administering to a subject in need thereof an effective amount of a vitamin D compound of the invention, such that uveitis is prevented or treated in the subject.

In one embodiment, the invention provides a method as described above, further comprising identifying a subject in need of prevention or treatment for uveitis. In another embodiment, the invention provides a method as described above, further comprising the step of obtaining the vitamin D compound of the invention. In one embodiment of the methods described herein, the subject is a mammal. In a further embodiment, the subject is a human.

In another embodiment, the invention provides a method described herein wherein the vitamin D compound of the invention is formulated in a pharmaceutical composition together with a pharmaceutically acceptable diluent or carrier.

In another aspect, the invention provides a pharmaceutical formulation comprising a vitamin D compound of the invention and a pharmaceutically acceptable carrier for use in the prevention and/or treatment of uveitis.

In yet another aspect, the invention provides a packaged pharmaceutical formulation comprising a vitamin D compound of the invention and a pharmaceutically acceptable carrier packaged with instructions for use in the prevention and/or treatment of uveitis.

The invention provides a kit containing a vitamin D compound of the invention together with instructions directing administration of said compound to a patient in need of treatment and/or prevention of uveitis thereby to treat and/or prevent uveitis in said patient.

In one embodiment, the invention provides for the use, method, formulation, compound or kit, wherein the vitamin D compound of the invention is administered separately, sequentially or simultaneously in separate or combined pharmaceutical formulations with a second medicament for the treatment of uveitis.

In one embodiment, the vitamin D compound of the invention is a vitamin D₃ compound of formula I:

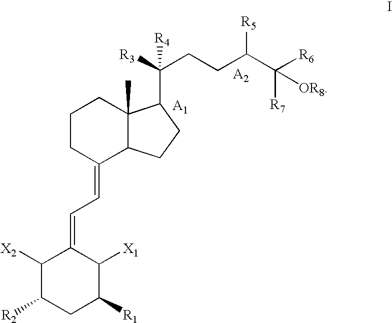

wherein: A₁ is single or double bond; A₂ is a single, double or triple bond; X₁ and X₂ are each independently H₂ or ═CH₂, provided X₁ and X₂ are not both ═CH₂; R₁ and R₂ are each independently OC(O)C₁-C₄ alkyl, OC(O)hydroxyalkyl, or OC(O)haloalkyl; R₃, R₄ and R₅ are each independently hydrogen, C₁-C₄ alkyl, hydroxyalkyl, or haloalkyl, with the understanding that R₅ is absent when A₂ is a triple bond, or R₃ and R₄ taken together with C₂₀ form C₃-C₆ cycloalkyl; R₆ and R₇ are each independently alkyl or haloalkyl; and R₈ is H, C(O)C₁-C₄ alkyl, C(O)hydroxyalkyl, or C(O)haloalkyl; and pharmaceutically acceptable esters, salts, and prodrugs thereof.

In one embodiment, the compounds of formula I are as described above provided that when A₁ is a single bond, R₃ is hydrogen and R₄ is methyl, then A₂ is a double or triple bond.

In another embodiment, the vitamin D compound of the invention is a vitamin D₃ compound of formula I-a:

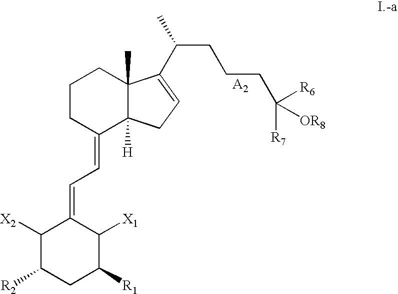

wherein (in formula I above, R₃ is H, R₄ is methyl, A₁ is a double bond,) R₅ is H (or absent if A₂ is a triple bond), and A₂, X₁, X₂, R₁, R₂, R₆, R₇, and R₈ are previously described.

In yet another embodiment, the vitamin D compound of the invention is a vitamin D₃ compound of formula I-b:

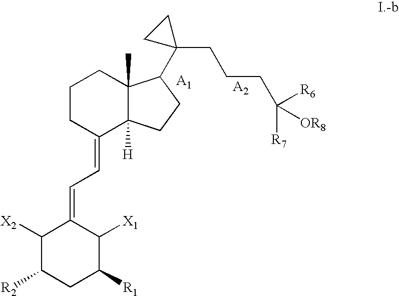

wherein (in formula I above, R₃ and R₄ taken together with C-20 form cyclopropyl), R₅ is H (or absent if A₂ is a triple bond), and A₁, A₂, X₁, X₂, R₁, R₂, R₆, R₇, and R₈ are previously described.

### DETAILED DESCRIPTION OF THE INVENTION

1. DEFINITIONS

Before further description of the present invention, and in order that the invention may be more readily understood, certain terms are first defined and collected here for convenience.

The term “administration” or “administering” includes routes of introducing the vitamin D₃ compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the vitamin D₃ compound can be coated with or disposed in a selected material to protect it from natural conditions which may detrimentally effect its ability to perform its intended function. The vitamin D₃ compound can be administered alone, or in conjunction with either another agent as described above or with a pharmaceutically-acceptable carrier, or both. The vitamin D₃ compound can be administered prior to the administration of the other agent, simultaneously with the agent, or after the administration of the agent. Furthermore, the vitamin D₃ compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “autoimmune disease” or “autoimmune disorder” refers to the condition where the immune system attacks the host's own tissue(s). In an autoimmune disease, the immune tolerance system of the patient fails to recognize self antigens and, as a consequence of this loss of tolerance, brings the force of the immune system to bear on tissues which express the antigen. Autoimmune disorders include, but are not limited to, type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitis, uveoretinitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

The language “biological activities” of vitamin D₃ includes all activities elicited by vitamin D₃ compounds in a responsive cell. It includes genomic and non-genomic activities elicited by these compounds (Gniadecki R. and Calverley M. J. (1998) Pharmacology & Toxicology 82: 173-176; Bouillon, R. et al. (1995) Endocrinology Reviews 16(2):206-207; Norman A. W. et al. (1992) J. Steroid Biochem Mol. Biol 41:231-240; Baran D. T. et al. (I991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient treat a vitamin D₃ associated state or to modulate ILT3 expression in a cell. An effective amount of vitamin D₃ compound may vary according to factors such as the disease state, age, and weight of the subject, and the ability of the vitamin D₃ compound to elicit a desired response in the subject. Dosage regimens may be adjusted to provide the optimum therapeutic response. An effective amount is also one in which any toxic or detrimental effects (e.g., side effects) of the vitamin D₃ compound are outweighed by the therapeutically beneficial effects.

A therapeutically effective amount of vitamin D₃ compound (i.e., an effective dosage) may range from about 0.001 to 30 μg/kg body weight, preferably about 0.01 to 25 μg/kg body weight, more preferably about 0.1 to 20 μg/kg body weight, and even more preferably about 1 to 10 μg/kg, 2 to 9 μg/kg, 3 to 8 μg/kg, 4 to 7 μg/kg, or 5 to 6 μg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a vitamin D₃ compound can include a single treatment or, preferably, can include a series of treatments. In one example, a subject is treated with a vitamin D₃ compound in the range of between about 0.1 to 20 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a vitamin D₃ compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The language “genomic” activities or effects of vitamin D₃ is intended to include those activities mediated by the nuclear receptor for 1α, 25(OH)₂D₃ (VD₃R), e.g., transcriptional activation of target genes.

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hormone secretion” is art-recognized and includes activities of vitamin D₃ compounds that control the transcription and processing responsible for secretion of a given hormone e.g., a parathyroid hormone (PTH) of a vitamin D₃ responsive cell (Bouillon, R. et al. (1995) Endocrine Reviews 16(2):235-237).

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

The term “immune response” includes T and/or B cell responses, e.g., cellular and/or humoral immune responses. The claimed methods can be used to reduce both primary and secondary immune responses. The immune response of a subject can be determined by, for example, assaying antibody production, immune cell proliferation, the release of cytokines, the expression of cell surface markers, cytotoxicity, and the like.

The terms “immunological tolerance” or “tolerance to an antigen” or “immune tolerance” include unresponsiveness to an antigen without the induction of a prolonged generalized immune deficiency. Consequently, according to the invention, a tolerant host is capable of reacting to antigens other than the tolerizing antigen. Tolerance represents an induced depression in the response of a subject that, had it not been subjected to the tolerance-inducing procedure, would be competent to mount an immune response to that antigen. In one embodiment of the invention, immunological tolerance is induced in an antigen-presenting cell, e.g., an antigen-presenting cell derived from the myeloid or lymphoid lineage, dendritic cells, monocytes and macrophages.

The language “immunosuppressive activity” refers to the process of inhibiting a normal immune response. Included in this response is when T and/or B clones of lymphocytes are depleted in size or suppressed in their reactivity, expansion or differentiation. Immunosuppressive activity may be in the form of inhibiting or blocking an immune response already in progress or may involve preventing the induction of an immune response. The functions of activated T cells may be inhibited by suppressing immune cell responses or by inducing specific tolerance, or both. Immunosuppression of T cell responses is generally an active, non-antigen-specific, process that requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

The language “improved biological properties” refers to any activity inherent in a compound of the invention that enhances its effectiveness in vivo. In a preferred embodiment, this term refers to any qualitative or quantitative improved therapeutic property of a vitamin D₃ compound, such as reduced toxicity, e.g., reduced hypercalcemic activity.

The language “inhibiting the growth” of the neoplasm includes the slowing, interrupting, arresting or stopping its growth and metastases and does not necessarily indicate a total elimination of the neoplastic growth.

The phrase “inhibition of an immune response” is intended to include decreases in T cell proliferation and activity, e.g., a decrease in IL₂, interferon-γ, GM-CSF synthesis and secretion (Lemire, J. M. (1992) J. Cell Biochemistry 49:26-31, Lemire, J. M. et al. (1994) Endocrinology 135 (6): 2813-2821; Bouillon, R. et al. (1995) Endocine Review 16 (2):231-32).

The term “isomers” or “stereoisomers” refers to compounds which have identical chemical constitution, but differ with regard to the arrangement of the atoms or groups in space.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

The language “non-genomic” vitamin D₃ activities include cellular (e.g., calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989) J. Biol. Chem. 264:20403-20406; Wali R. K. et al. (1992) Endocrinology 131:1125-1133; Wali R. K. et al. (1992) Am. J Physiol. 262:G945-G953; Wali R. K. et al. (1990) J. Clin. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinologyl 18:2300-2304. Changes in calcium activity and second messenger systems are well known in the art and are extensively reviewed in Bouillion, R. et al. (1995) Endocrinology Review 16(2): 200-257; the description of which is incorporated herein by reference.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “reduced toxicity” is intended to include a reduction in any undesired side effect elicited by a vitamin D₃ compound when administered in vivo, e.g., a reduction in the hypercalcemic activity.

The term “secosteroid” is art-recognized and includes compounds in which one of the cyclopentanoperhydro-phenanthrene rings of the steroid ring structure is broken. 1α,25(OH)₂D₃ and analogs thereof are hormonally active secosteroids. In the case of vitamin D₃, the 9-10 carbon-carbon bond of the B-ring is broken, generating a seco-B-steriod. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

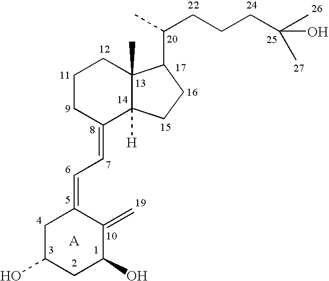

In the formulas presented herein, the various substituents on ring A are illustrated as joined to the steroid nucleus by one of these notations: a dotted line ( - - - ) or ( [symbol] ) indicating a substituent which is in the β-orientation (i.e. , above the plane of the ring), a wedged solid line ( [symbol] ) indicating a substituent which is in the α-orientation (i.e., below the plane of the molecule), or a wavy line ( [symbol] ) indicating that a substituent may be either above or below the plane of the ring. In regard to ring A, it should be understood that the stereochemical convention in the vitamin D field is opposite from the general chemical field, wherein a dotted line indicates a substituent on Ring A which is in an α-orientation (i.e., below the plane of the molecule), and a wedged solid line indicates a substituent on ring A which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3β-hydroxyl groups. In other words, carbons 1 and 3 of the A ring are said to be “chiral carbons” or “carbon centers.” Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

Also, throughout the patent literature, the A ring of a vitamin D compound is often depicted in generic formulae as any one of the following structures:

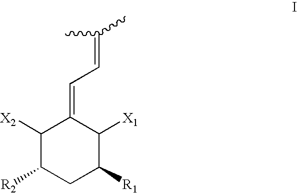

wherein X₁ and X₂ are defined as H (or H₂) or ═CH₂; or

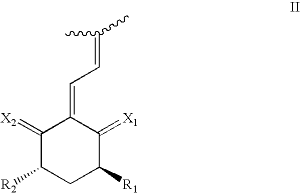

wherein X₁ and X₂ are defined as H₂ or CH₂. Although there does not appear to be any set convention, it is clear that one of ordinary skill in the art understands either formula I or II to represent an A ring in which, for example, X₁ is ═CH₂ and X₂ is defined as H₂, as follows:

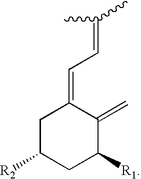

For purposes of the instant invention, formula I will be used in all generic structures.

The term “sulfhydryl” or “thiol” means —SH.

The term “subject” includes organisms which are capable of suffering from a vitamin D₃ associated state or who could otherwise benefit from the administration of a vitamin D₃ compound of the invention, such as human and non-human animals. Preferred human animals include human patients suffering from or prone to suffering from a vitamin D₃ associated state, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The phrases “systemic administration,” “administered systemically”, “peripheral administration” and “administered peripherally” as used herein mean the administration of a vitamin D₃ compound(s), drug or other material, such that it enters the patient's system and, thus, is subject to metabolism and other like processes, for example, subcutaneous administration.

The language “therapeutically effective anti-neoplastic amount” of a vitamin D₃ compound of the invention refers to an amount of an agent which is effective, upon single or multiple dose administration to the patient, in inhibiting the growth of a neoplastic vitamin D₃-responsive cells, or in prolonging the survivability of the patient with such neoplastic cells beyond that expected in the absence of such treatment.

The language “transplant rejection” refers to an immune reaction directed against a transplanted organ(s) from other human donors (allografts) or from other species such as sheep, pigs, or non-human primates (xenografts). Therefore, the method of the invention is useful for preventing an immune reaction to transplanted organs from other human donors (allografts) or from other species (xenografts). Such tissues for transplantation include, but are not limited to, heart, liver, kidney, lung, pancreas, pancreatic islets, bone marrow, brain tissue, cornea, bone, intestine, skin, and hematopoietic cells. Also included within this definition is “graft versus host disease” of “GVHD,” which is a condition where the graft cells mount an immune response against the host. Therefore, the method of the invention is useful in preventing graft versus host disease in cases of mismatched bone marrow or lymphoid tissue transplanted for the treatment of acute leukemia, aplastic anemia, and enzyme or immune deficiencies, for example. The term “transplant rejection” also includes disease symptoms characterized by loss of organ function. For example, kidney rejection would be characterized by a rising creatine level in blood. Heart rejection is characterized by an endomyocardial biopsy, while pancreas rejection is characterized by rising blood glucose levels. Liver rejection is characterized by the levels of transaminases of liver origin and bilirubin levels in blood. Intestine rejection is determined by biopsy, while lung rejection is determined by measurement of blood oxygenation.

By “uveitis” it is meant conditions comprising inflammation of the eye, in particular the uveal tract (iris, ciliary body, choroid) with or without additional inflammation of the retina, optic nerve and vitreous, including but not limited to anterior, intermediate, posterior uveitis and panuveitis and in acute, sub-acute, chronic or recurrent forms.

The term “VDR” is intended to include members of the type II class of steroid/thyroid superfamily of receptors (Stunnenberg, H. G. (1993) Bio Essays 15(5):309-15), which are able to bind and transactivate through the vitamin D response element (VDRE) in the absence of a ligand (Damm et al. (1989) Nature 339:593-97; Sap et al. Nature 343:177-180).

The term “VDRE” refers to DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al. (1991) Cell 67:1251-1266; Bugge et al. (1992) EMBO J. 11:1409-1418; Kliewer et al. (1992) Nature 355:446-449; Leid et al. (1992) EMBO J. 11:1419-1435; Zhang et al. (1992) Nature 355:441-446).

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

2. VITAMIN D3 COMPOUNDS OF THE INVENTION

A prominent feature of the vitamin D₃ compounds used in the methods of the invention is acylation at the I and 3 positions on the A ring of the compounds. Certain 1,3-diacyl vitamin D₃ compounds are described in U.S. Pat. No. 5,976,784 to DeLuca et al.

The acylated vitamin D₃ compounds of formula I above exert a full spectrum of 1,25(OH)₂D₃ biological activities such as binding to the specific nuclear receptor VDR, suppression of the increased parathyroid hormone levels in 5,6-nephrectomized rats, suppression of INF-γ release in MLR cells, stimulation of HL-60 leukemia cell differentiation and inhibition of solid tumor cell proliferation. It is well known that in vivo and in cellular cultures 1,25-(OH)₂D₃ undergoes a cascade of metabolic modifications initiated by the influence of 24R-hydroxylase enzyme. First 24R-hydroxy metabolite is formed, which is oxydized to 24-keto intermediate, and then 23S-hydroxylation and fragmentation produce the fully inactive calcitroic acid.

It has been discovered that 1,3-diacylated compounds of the invention have unexpected and/or superior properties as compared to corresponding 1,3-dihydroxy compounds. For example, 1,3-Di-O-acetyl-1,25-dihydroxy-16,23Z-diene-26,27-hexafluoro-19-nor-cholecalciferol (2), 1,3-Di-O-acetyl-1,25-Dihydroxy-16-ene-23-yne-26,27-hexafluoro-19-nor-cholecalciferol (4) and 1,3,25-Tri-O-acetyl-1,25-Dihydroxy-16-ene-23-yne-26,27-hexafluoro-19-nor-cholecalciferol (5) have a significantly higher maximum tolerated dose and improved activity when compared with the corresponding 5 dihydroxy compounds, 1,25-dihydroxy-16,23Z-diene-26,27-hexafluoro-19-nor-cholecalciferol (1) and 1,25-dihydroxy-16-ene-23-yne-26,27-hexafluoro-19-nor-cholecalciferol (3).

Thus, in one embodiment, the methods provided by the invention make use of a vitamin D₃ compound of formula I:

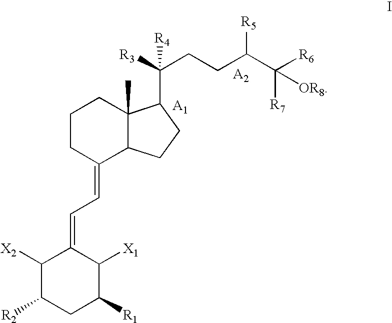

wherein:

A₁ is single or double bond;

A₂ is a single, double or triple-bond;

X₁ and X₂ are each independently H₂ or ═CH₂, provided X₁ and X₂ are not both ═CH₂;

R₁ and R₂ are each independently OC(O)C₁-C₄ alkyl, OC(O)hydroxyalkyl, or OC(O)haloalkyl;

R₃, R₄ and R₅ are each independently hydrogen, C₁-C₄ alkyl, hydroxyalkyl, or haloalkyl, with the understanding that R₅ is absent when A₂ is a triple bond, or R₃ and R₄ taken together with C₂₀ form C₃-C₆ cycloalkyl;

R₆ and R₇ are each independently alkyl or haloalkyl; and
R₈ is H, C(O)C₁-C₄ alkyl, C(O)hydroxyalkyl, or C(O)haloalkyl;
provided that when A₁ is single bond, R₃ is hydrogen and R₄ is methyl, then A₂ is a double or triple bond; and

pharmaceutically acceptable esters, salts, and prodrugs thereof.

In one embodiment of the invention, X₁ is H₂ and X₂ is ═CH₂. In another embodiment, X₁ and X₂ are H₂. In another embodiment, A₁is a single bond. In another embodiment, A₁ is a double bond. In another embodiment, A₁ is a triple bond.

In a preferred embodiment, R₃ is hydrogen and R₄ is C₁-C₄ alkyl, preferably methyl. In another preferred embodiment, R₃ and R_{4,} taken together with C₂₀, form C₃-C₆ cycloalkyl. In a preferred embodiment, R₃ and R_{4,} taken together with C₂₀, form cyclopropyl.

In a preferred embodiment, R₁ and R₂ are each independently OC(O)C₁-C₄ alkyl, preferably OC(O)CH₃.

In a preferred embodiment, R₆ and R₇ are each independently alkyl or haloalkyl, preferably methyl, ethyl, or trifluoromethyl.

In a preferred embodiment, R₈ is H or C(O)C₁-C₄ alkyl.

Certain embodiments fo the invention are directed to the use of 1,3-acylated, 26,27-haloakly vitamin D₃ compounds. Such compounds are represented by the formula I-c:

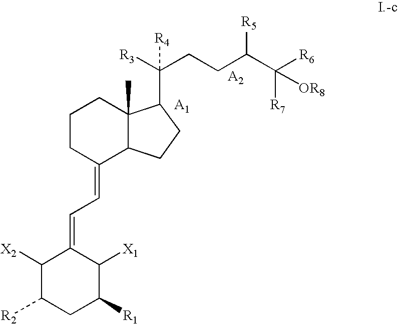

wherein:

A₁ is single or double bond;

A₂ is a single, double or triple bond,

X₁ and X₂ are each independently H₂ or CH₂, provided X₁ and X₂ are not both CH₂;

R₁ and R₂ are each independently OC(O)C₁-C₄ alkyl, OC(O)hydroxyalkyl, or OC(O)haloalkyl;

R₃, R₄ and R₅ are each independently hydrogen, C₁-C₄ alkyl, hydroxyalkyl, or haloalkyl, or R₃ and R₄ taken together with C₂₀ form C₃-C₆ cylcoalkyl;

R₆ and R₇ are each independently haloalkyl; and

R₈ is H, OC(O)C₁-C₄ alkyl, OC(O)hydroxyalkyl, or OC(O)haloalkyl; and

pharmaceutically acceptable esters, salts, and prodrugs thereof. In preferred embodiments, R₆ and R₇ are each independently trihaloalkyl, especially trifluoromethyl.

In another embodiment of the invention, R₁ and R₂ are OC(O)CH₃, R₃ is H, R₄ is methyl, R₅ is H (or absent if A₂ is a triple bond), as shown in formula I-a.

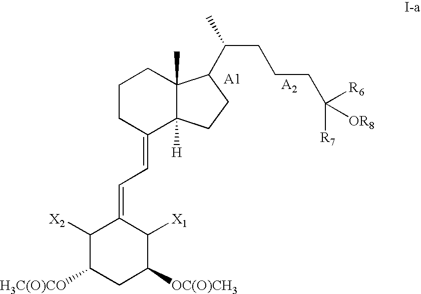

In a preferred embodiment, A₁ is a double bond, and X₁ is ═CH₂ and X₂ is H₂. When A₂ is a triple bond, it is preferred that R₈ is H or C(O)CH₃, and R₆ and R₇ are alkyl or haloalkyl. It is preferred that the alkyl group is methyl and the haloalkyl group is trifluoroalkyl, preferably trifluoromethyl. When A₂ is a double bond, it is preferred that R₈ is H or C(O)CH₃, and R₆ and R₇ are alkyl, preferably methyl. It is also preferred that R₆ and R₇ are independently alkyl and haloalkyl. When A₂ is a single bond, it is preferred that R₈ is H or C(O)CH₃, and R₆ and R₇ are alkyl, preferably methyl.

In a preferred embodiment, A₁ is a double bond, and X₁ and X₂ are each H₂. When A₂ is a triple bond, it is preferred that R₈ is H or C(O)CH₃, and R₆ and R₇ are alkyl or haloalkyl. It is preferred that the alkyl group is methyl or ethyl and the haloalkyl group is trifluoroalkyl, preferably trifluoromethyl. When A₂ is a double bond, it is preferred that R₈ is H or C(O)CH₃, and R₆ and R₇ are haloalkyl, preferably trifluoroalkyl, preferably trifluoromethyl. When A₂ is a single bond, it is preferred that R₈ is H or C(O)CH₃, and R₆ and R₇ are alkyl, preferably methyl.

In another embodiment of the invention of formula (I), R₁ and R₂ are OC(O)CH₃, A₁ is a single bond, and A₂ is a single, double or triple bond, except that when R₃ is H and R₄ is methyl, A₂ is a double or triple bond. In a preferred embodiment, R₃ is H, R₄ is methyl, R₅ is absent, R₈ is H or C(O)CH₃, and R₆ and R₇ are alkyl, preferably methyl.

Preferred compounds of the present invention are summarized in Table 1 and include the following: 1,3-Di-O-acetyl-1,25-dihydroxy-16,23Z-diene-26,27-hexafluoro-19-nor-cholecalciferol (2), 1,3-Di-O-acetyl-1,25-Dihydroxy-16-ene-23-yne-26,27-hexafluoro-19-nor-cholecalciferol (4), 1,3,25-Tri-O-acetyl-1,25-Dihydroxy-16-ene-23-yne-26,27-hexafluoro-19-nor-cholecalciferol (5), 1,3-Di-O-acetyl-1,25-dihydroxy-16-ene-23-yne-cholecalciferol (7),1,3-Di-O-acetyl-1,25-dihydroxy-16,23E-diene-cholecalciferol (9), 1,3-Di-O-acetyl-1,25-dihydroxy-16-ene-cholecalciferol (11), 1,3,25-Tri-O-acetyl-1,25-dihydroxy-16-ene-23-yne-26,27-hexafluoro-cholecalciferol (13), 1,3-Di-O-acetyl-1,25-dihydroxy-16-ene-23-yne-26,27-hexafluoro-cholecalciferol (14), 1,3-Di-O-acetyl-1,25-dihydroxy-16,23E-diene-25R,26-trifluoro-cholecalciferol (16), 1,3-Di-O-acetyl-1,25-dihydroxy-16-ene-19-nor-cholecalciferol (18), 1,3-Di-O-Acetyl-1,25-dihydroxy-16-ene-23-yne-19-nor-cholecalciferol (20), 1,3-Di-O-acetyl-1,25-dihydroxy-16-ene-23-yne-26,27-bishomo-19-nor-cholecalciferol (22) and 1,3-Di-O-acetyl-1,25-dihydroxy-23-yne-cholecalciferol (41).

TABLE 1

| | |
|---|---|
| | I-a |
| 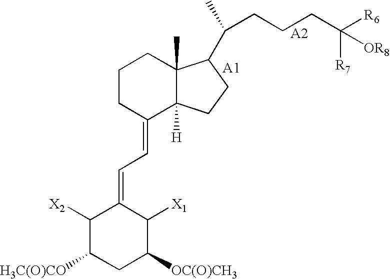 | |

| Compound | X₁ | X₂ | A₁ | A₂ | R₆ | R₇ | R₈ |
|---|---|---|---|---|---|---|---|
| (2)^{a} | H₂ | H₂ | ═ | ═ | CF₃ | CF₃ | H |
| (4) | H₂ | H₂ | ═ | ≡ | CF₃ | CF₃ | H |
| (5) | H₂ | H₂ | ═ | ≡ | CF₃ | CF₃ | C(O)CH₃ |
| (7) | ═CH₂ | H₂ | ═ | ≡ | CH₃ | CH₃ | H |
| (9) | ═CH₂ | H₂ | ═ | ═ | CH₃ | CH₃ | H |
| (11) | ═CH₂ | H₂ | ═ | — | CH₃ | CH₃ | H |
| (13) | ═CH₂ | H₂ | ═ | ≡ | CF₃ | CF₃ | C(O)CH₃ |
| (14) | ═CH₂ | H₂ | ═ | ≡ | CF₃ | CF₃ | H |
| (16) | ═CH₂ | H₂ | ═ | ═ | CF₃ | CH₃ | H |
| (18) | H₂ | H₂ | ═ | — | CH₃ | CH₃ | H |
| (20) | H₂ | H₂ | ═ | ≡ | CH₃ | CH₃ | H |
| (22) | H₂ | H₂ | ═ | ≡ | CH₂CH₃ | CH₂CH₃ | H |
| (41) | ═CH₂ | H₂ | — | ≡ | CH₃ | CH₃ | H |

^{a}Z olefin

In another embodiment of the invention, R₁ and R₂ are each OC(O)CH₃, and R₃ and R₄ taken together with C₂₀ form cyclopropyl, and R₅ is H (or absent if A₂ is a triple bond), as shown in formula I-b.

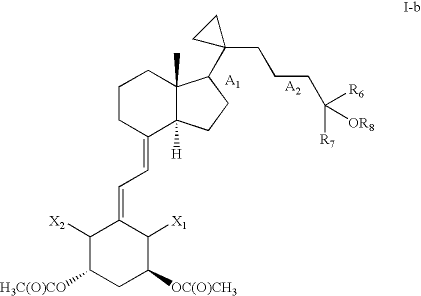

In a preferred embodiment, X₁ is ═CH₂ and X₂ is H₂. When A₁ is a single bond, and A₂ is a triple bond, it is preferred that R₈ is H or C(O)CH₃, and R₆ and R₇ are alkyl, preferably methyl. When A₁ is a single bond, and A₂ is a single bond, it is preferred that R₈ is H or C(O)CH₃, and R₆ and R₇ are alkyl, preferably methyl. When A₁ is a double bond, and A₂ is a single bond, it is preferable that R₈ is H or C(O)CH₃, and R₆ and R₇ are alkyl, preferably methyl.

In another preferred embodiment, X₁ and X₂ are each H₂. When A₁ is a single bond, and A₂ is a triple bond, it is preferred that R₈ is H or C(O)CH₃, and R₆ and R₇ are alkyl or haloalkyl. It is preferred that the alkyl group is methyl, and the haloalkyl group is trifluoroalkyl, preferably trifluoromethyl. When A₁ is a single bond, and A₂ is a double bond, it is preferred that R₈ is H or C(O)CH₃, R₆ and R₇ are haloalkyl, preferably trifluoroalkyl, preferably trifluoromethyl. When A₁ is a double bond, and A₂ is a single bond, it is preferred that R₈ is H or C(O)CH₃, R₆ and R₇ are alkyl, preferably methyl.

Preferred compounds of the present invention are summarized in Table 2 and include the following: 1,3-Di-O-acetyl-1,25-dihydroxy-20-cyclopropyl-23-yne-19-nor-cholecalciferol (24), 1,3,25-Tri-O-acetyl-1,25-dihydroxy-20-cyclopropyl-23-yne-26,27-hexafluoro-19-nor-cholecalciferol (26), 1,3-Di-O-acetyl-1,25-dihydroxy-20-cyclopropyl-23-yne-26,27-hexafluoro-19-nor-cholecalciferol (27), 1,3-Di-O-acetyl-1,25-dihydroxy-20-cyclopropyl-23-yne-cholecalciferol (29), 1,3-Di-O-acetyl-1,25-dihydroxy-20-cyclopropyl-23E-ene-26,27-hexafluoro-19-nor-cholecalciferol (31), 1,3-Di-O-acetyl-1,25-dihydroxy-20-cyclopropyl-23Z-ene-26,27-hexafluoro-19-nor-cholecalciferol (33), 1,3-Di-O-acetyl-1,25-dihydroxy-20-cyclopropyl-cholecalciferol (35) (“Compound A”), 1,3-Di-O-acetyl-c1,25-dihydroxy-16-ene-20-cyclopropyl-19-nor-cholecalciferol (37), and 1,3-Di-O-acetyl-1 c,25-hydroxy-16-ene-20-cyclopropyl-cholecalciferol (39).

TABLE 2

| | |
|---|---|
| | I-b |
| 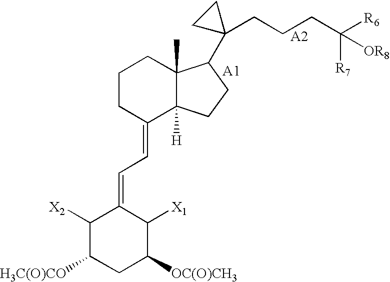 | |

| Compound | X₁ | X₂ | A₁ | A₂ | R₆ | R₇ | R₈ |
|---|---|---|---|---|---|---|---|
| (24) | H₂ | H₂ | — | ≡ | CH₃ | CH₃ | H |
| (27) | H₂ | H₂ | — | ≡ | CF₃ | CF₃ | H |
| (26) | H₂ | H₂ | — | ≡ | CF₃ | CF₃ | C(O)CH₃ |
| (29) | ═CH₂ | H₂ | — | ≡ | CH₃ | CH₃ | H |
| (31) | H₂ | H₂ | — | ═ | CF₃ | CF₃ | H |
| (33)^{a} | H₂ | H₂ | — | ═ | CF₃ | CF₃ | H |
| (35) | ═CH₂ | H₂ | — | — | CH₃ | CH₃ | H |
| (37) | H₂ | H₂ | ═ | — | CH₃ | CH₃ | H |
| (39) | ═CH₂ | H₂ | ═ | — | CH₃ | CH₃ | H |

^{a}Z olefin.

An example of a preferred compound is 1,3-Di-O-acetyl-1,25-dihydroxy-20-cyclopropyl-cholecalciferol (referred to as “Compound A” in examples) having the formula:

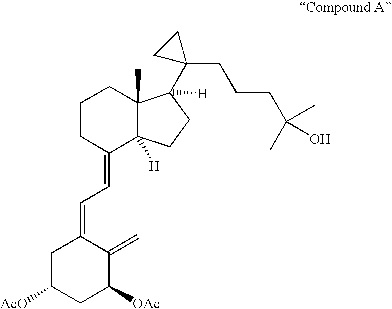

The invention also embraces use of esters and salts of Compound A. Esters include pharmaceutically acceptable labile esters that may be hydrolysed in the body to release Compound A. Salts of Compound A include adducts and complexes that may be formed with alkali and alkaline earth met al ions and met al ion salts such as sodium, potassium and calcium ions and salts thereof such as calcium chloride, calcium malonate and the like. However, although Compound A may be administered as a pharmaceutically acceptable salt or ester thereof, preferably Compound A is employed as is i.e., it is not employed as an ester or a salt thereof.

The structures of some of the compounds of the invention include asymmetric carbon atoms. Accordingly, the isomers arising from such asymmetry (e.g., all enantiomers and diastereomers) are included within the scope of the invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

3. PHARMACEUTICAL COMPOSITIONS

The invention also provides a pharmaceutical composition, comprising an effective amount of a vitamin D₃ compound of formula I or otherwise described herein and a pharmaceutically acceptable carrier. In a further embodiment, the effective amount is effective to treat a vitamin D₃ associated state, as described previously.

In an embodiment, the vitamin D₃ compound is administered to the subject using a pharmaceutically-acceptable formulation, e.g., a pharmaceutically-acceptable formulation that provides sustained delivery of the vitamin D₃ compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those vitamin D₃ compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and anfioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) met al chelating agents, such as citric acid, ethylenediamine tetraacetic acid (EDTA), sorbitol, tartaric acid, phosphoric acid, and the like.

Compositions containing a vitamin D₃ compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these compositions include the step of bringing into association a vitamin D₃ compound(s) with the carrier and, optionally, one or more accessory ingredients. In general, the formulations are prepared by uniformly and intimately bringing into association a vitamin D₃ compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D₃ compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed-as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D₃ compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D₃ compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof Pharmaceutical compositions of the invention for rectal or vaginal administration may be presented as a suppository, which may be prepared by mixing one or more vitamin D₃ compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D₃ compound(s) include powders, sprays, ointments, pastes, creams, lotions, gels, solutions, patches and inhalants. The active vitamin D₃ compound(s) may be mixed under sterile conditions with a pharmaceutically-acceptable carrier, and with any preservatives, buffers, or propellants which may be required.

The ointments, pastes, creams and gels may contain, in addition to vitamin D₃ compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D₃ compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D₃ compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D₃ compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Pharmaceutical compositions of the invention suitable for parenteral administration comprise one or more vitamin D₃ compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D₃ compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D₃ compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D₃ compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the vitamin D₃ compound for the present invention is the maximum that a patient can tolerate and not develop serious hypercalcemia. Preferably, the vitamin D₃ compound of the present invention is administered at a concentration of about 0.001 μg to about 100 μg per kilogram of body weight, about 0.001-about 10 μg/kg or about 0.001 μg-about 100 μg/kg of body weight. Ranges intermediate to the above-recited values are also intended to be part of the invention.

### EXEMPLIFICATION OF THE INVENTION

The invention is further illustrated by the following examples which should in no way should be construed as being further limiting.

### SYNTHESIS OF COMPOUNDS OF THE INVENTION

Experimental

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25620 C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 ml/min. The results are summarized in Table 1 for examples 1-10 and 19 (C₂₀-natural), and Table 2 for examples 11-18 (C₂₀-cyclopropyl).

### EXAMPLE 1

### Synthesis of 1,3-Di-O-acetyl-1,25-dihydroxy-16,23Z-diene-26,2 7-hexafluoro-19-nor-cholecalciferol (2)

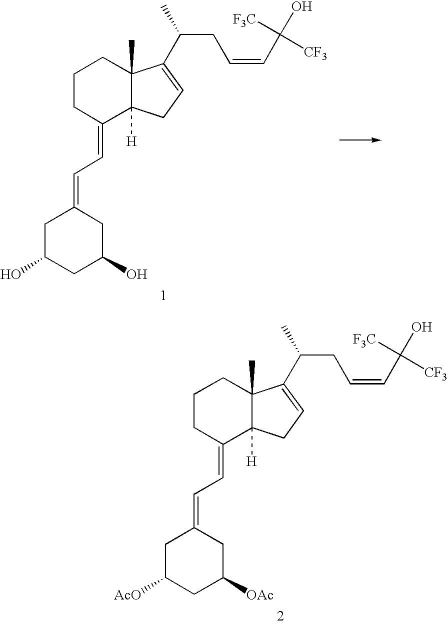

The starting material 1,25-dihydroxy-16,23Z-diene-26,27-hexafluoro-19-nor-cholecalciferol (1) can be prepared as described in U.S. Pat. No. 5,428,029 to Doran et al. 3 mg of 1,25-dihydroxy-16,23Z-diene-26,27-hexafluoro-19-nor-cholecalciferol (1) was dissolved in 0.8 ml of pyridine, cooled to ice-bath temperature and 0.2 ml of acetic anhydride was added and maintained at that temperature for 16 h. Then the reaction mixture was diluted with 1 ml of water, stirred for 10 min in the ice bath and distributed between 5 ml of water and 20 ml of ethyl acetate. The organic layer was washed with 3×5 ml of water, once with 5 ml of saturated sodium hydrogen carbonate, once with 3 ml of brine then dried (sodium sulfate) and evaporated. The oily residue was taken up in 1:6 ethyl acetate-hexane and flash-chromatographed using a stepwise gradient of 1:6, 1:4 and 1:2 ethyl acetate-hexane. The column chromatography was monitored by TLC (1:4 ethyl acetate-hexane, spot visualization with phosphomolybdic acid spray), the appropriate fractions were pooled, evaporated, the residue taken up in methyl formate, filtered, then evaporated again to give 23.8 mg of the title compound (2) as-a colorless syrup; 400 MHz ¹H NMR δ 0.66 (3H, s), 0.90 (1H, m), 1.06 (3H, d, J=7.2 Hz), 1.51 (1H, m), 1.72-1.82 (3H,m), 1.9-2.1 (3H, m), 1.99 (3H, s) 2.04 (3H,s), 2.2-2.3 (3 m), 2.44-2.64 (6H, m), 2.78 (1H, m), 3.01 (1H, s), 5.10 (2H, m). 5.38 (1H, m), 5.43 (1H, d, J=12 Hz), 5.85 (1H, d, J=11.5 Hz), 5.97 (1H, dt, J=12 and 7.3Hz), 6.25 (1H, d,J=11.5 Hz).

### EXAMPLE 2

### Synthesis of 1,3-Di-O-acetyl-1,25-Dihydroxy-16-ene-23-yne-26,27-hexafluoro-19-nor-cholecalciferol (4) and 1,3,25-Tri-O-acetyl-1,25-Dihydroxy-16-ene-23-yne-26,27-hexafluoro-19-nor-cholecalciferol (5)

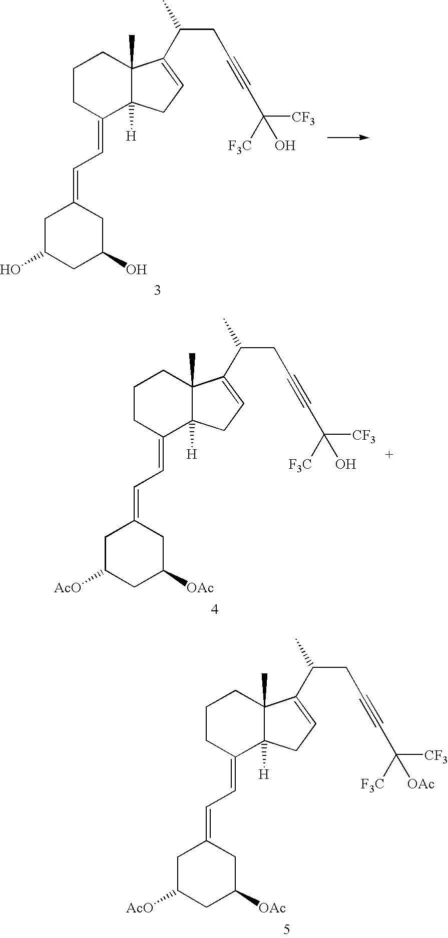

The starting material 1,25-dihydroxy-16-ene-23-yne-26,27-hexafluoro-19-nor-cholecalciferol (3) can be prepared as described in U.S. Pat. Nos. 5,451,574 and 5,612,328 to Baggiolini et al. 314 mg (0.619 mmole) of 1,25-dihydroxy-16-ene-23-yne-26,27-hexafluoro-19-nor-cholecalciferol (3) was dissolved in 1.5 ml of pyridine, cooled to ice-bath temperature, and 0.4 ml of acetic anhydride was added. The reaction mixture was kept at room temperature for 7 hours and then for 23 hours in a refrigerator. It was then diluted with 10 ml water and extracted with 30 ml of ethyl acetate. The organic extract was washed with water and brine, dried over sodium sulfate and evaporated. The residue was FLASH chromatographed on a 10×140 mm column with 1:6 and 1:4 ethyl acetate-hexane as the mobile phase to give 126 mg of 1,3-Di-O-acetyl-1,25-Dihydroxy-16-ene-23-yne-26,27-hexafluoro-19-nor-cholecalciferol (4), and 248 mg of 1,3,25-Tri-O-acetyl-1,25-Dihydroxy-16-ene-23-yne-26,27-hexafluoro-19-nor-cholecalciferol (5).

### EXAMPLE 3

### Synthesis of 1,3-Di-O-acetyl-1,25-dihydroxy-16-ene-23-yne-cholecalciferol (7)

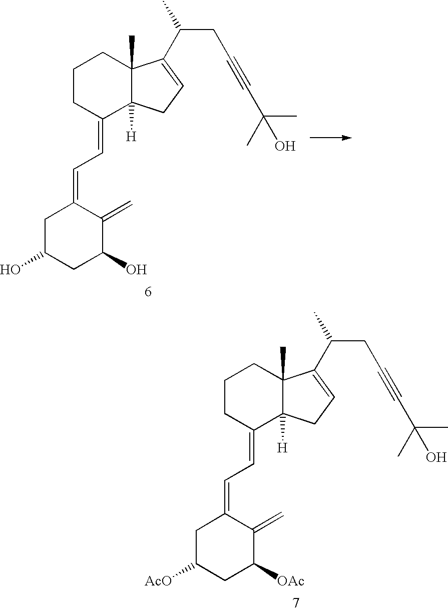

A 10-mL round-bottom flask was charged with 40 mg of 1,25-dihydroxy-16-ene-23-yne-cholecalciferol (6). This material was dissolved in 1 mL of pyridine. This solution was cooled in an ice bath then 0.3 mL of acetic anhydride was added. The solution was stirred for 30 min, then refrigerated overnight, diluted with water and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-5, 1:6 for fractions 6-13 and 1:4 ethyl acetate-hexane for fractions 14-20 (18 mL fractions). Fractions 14-19 contained the main band with Rf0.15 (TLC 1:4). Those fractions were pooled and evaporated to a colorless oil, 0.044 g. The material was taken up in methyl formate, filtered and evaporated to give a colorless, sticky foam, 0.0414 g of the title compound (7).

### EXAMPLE 4

### Synthesis of 1,3-Di-O-acetyl-1,25-dihydroxy-16,23E-diene-cholecalciferol (9)

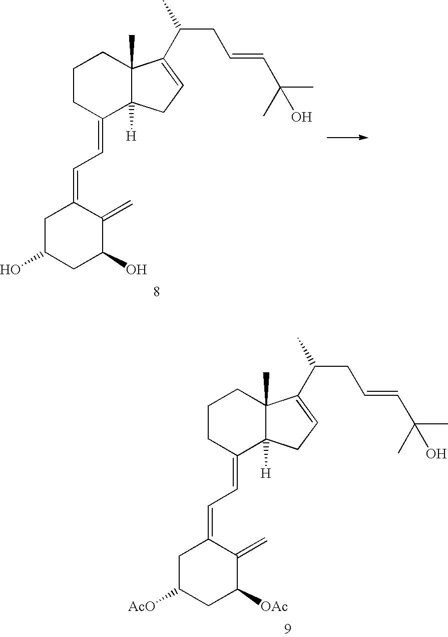

0.0468 g of 1,25-Dihydroxy-16,23E-diene-cholecalciferol (8) was dissolved in 1.5 mL of pyridine. This solution was cooled in an ice bath then refrigerated overnight, diluted with 10 mL of water while still immersed in the ice bath, stirred for 10 min and transferred to a separatory funnel with the aid of 10 mL of water and 40 mL of ethyl acetate. The organic layer was washed with 4×20 mL of water, 10 mL of brine passed through a plug of sodium sulfate and evaporated. The light brown, oily residue was taken up in 1:9 ethyl acetate-hexane then flash chromatographed on a 10×130 mm column using 1:9 ethyl acetate-hexane as mobile phase for fractions 1-3 (20 mL fractions), 1:6 for fractions 6-8 and 1:4 ethyl acetate-hexane for fractions 9-17 (18 mL each). Fractions 11-14 contained the main band with Rf 0.09 (TLC 1:4). Those fractions were pooled and evaporated to a colorless oil, 0.0153 g. This material was taken up in methyl formate, filtered and evaporated, to give 0.014 g of the title compound (9).

### EXAMPLE 5

### Synthesis of 1,3-Di-O-acetyl-1,25-dihydroxy-16-ene-cholecalciferol (11)

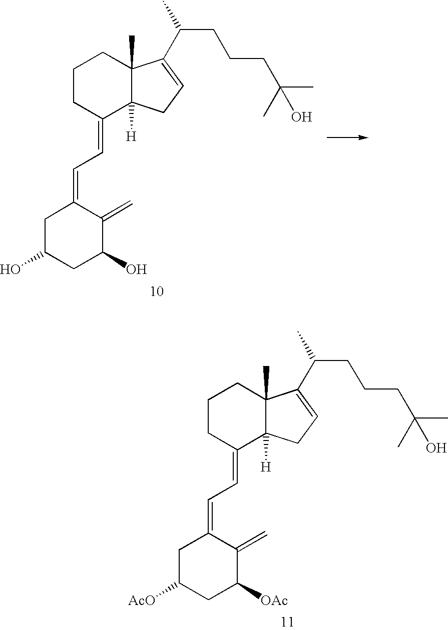

0.0774 g of 1,25-Dihydroxy-16-ene-cholecalciferol (10) was dissolved in 1.5 mL of pyridine. This solution was cooled in an ice bath then 0.3 mL of acetic anhydride was added. The solution was stirred, refrigerated overnight then diluted with 1 mL of water, stirred for 1 h in the ice bath and diluted with 30 mL of ethyl acetate and 15 mL of water. The organic layer was washed with 4×15 mL of water, once with 5 mL of brine then dried (sodium sulfate) and evaporated. The light brown, oily residue was taken up in 1:9 ethyl acetate-hexane then flash, chromatographed on a 10×130 mm column using 1:9 ethyl acetate-hexane as mobile phase for fraction 1 (20 mL fractions), 1:6 for fractions 2-7 and 1:4 ethyl acetate-hexane for fractions 8-13. Fractions 9-11 contained the main band with Rf 0.09 (TLC 1:4 ethyl acetate-hexane). Those fractions were pooled and evaporated to a colorless oil, 0.0354 g. This material was taken up in methyl formate, filtered and the solution evaporated, 0.027 g colorless film, the title compound (11).

### EXAMPLE 6

### Synthesis of 1,3,25-Tri-O-acetyl-1,25-dihydroxy-16-ene-23-yne-26,27-hexafluoro-cholecalciferol (13) and 1,3-Di-O-acetyl-1,25-dihydroxy-16-ene-23-yne-26,27-hexafluoro-cholecalciferol (14)

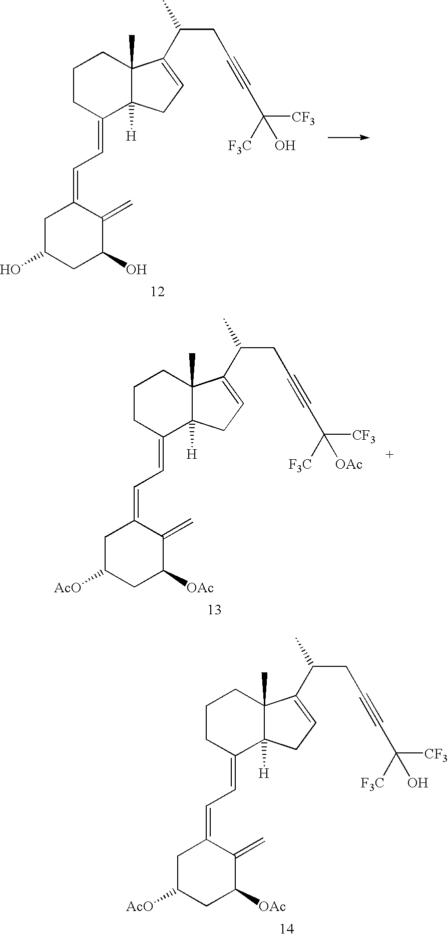

0.0291 g of 1,25-dihydroxy-16-ene-23-yne-26,27-hexafluoro-cholecalciferol (12) was dissolved in 1.5 mL of pyridine. This solution was cooled in an ice bath then 0.25 mL of acetic anhydride was added. The solution was stirred for 20 min and kept in a freezer overnight. The cold solution was diluted with 15 mL of water, stirred for 10 min, and diluted with 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×10 mm column using 1:6 ethyl acetate-hexane as mobile phase. Fractions 2-3 gave 72.3461−72.3285=0.0176 g. Evaporation of fractions 6-7 gave 0.0055 g. The residue of fractions 2-3 was taken up in methyl formate, filtered and evaporated to give 0.0107 g of the title triacetate (13). The residue of fractions 6-7 was taken up in methyl formate, filtered and evaporated to give 0.0049 g of diacetate (14).

### EXAMPLE 7

### Synthesis of 1,3-Di-O-acetyl-1,25-dihydroxy-16,23E-diene-25R,26-trifluoro-cholecalciferol (16)

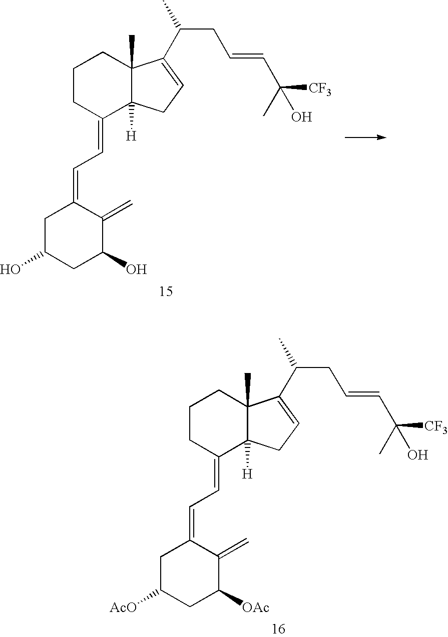

1.5 mL of 1,25-dihydroxy-16,23E-diene-25R,26-trifluoro-cholecalciferol (15) was dissolved in 1.5 mL of pyridine, cooled to ice-bath temperature and 0.4 mL of acetic anhydride was added. The mixture was then refrigerated. After two days the mixture was diluted with I mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The light brown, oily residue was taken up in 1:6 ethyl acetate-hexane then flash chromatographed on a 10×130 mm column using 1:6 ethyl acetate-hexane as mobile phase. Fractions 4-6 (TLC, 1:4) contained the main band (see TLC) These fractions were evaporated and gave 0.0726 g. This residue was taken up in methyl formate, filtered and evaporated, to give 0.0649 g of colorless foam, the title compound (16).

### EXAMPLE 8

### Synthesis of 1,3-Di-O-acetyl-1,25-dihydroxy-16-ene-19-nor-cholecalciferol (18)

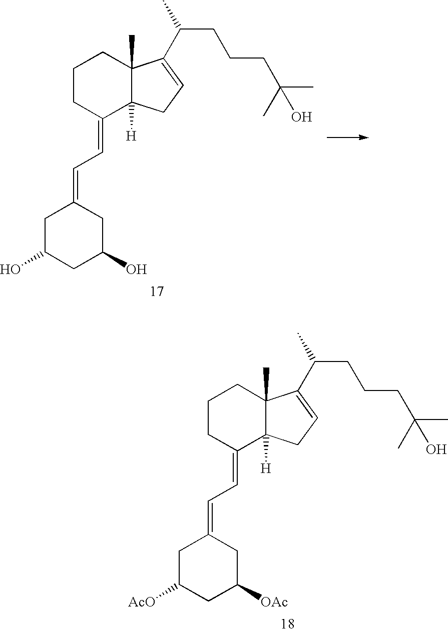

0.0535 g of 1,25-Dihydroxy-16-ene-19-nor-cholecalciferol (17) was dissolved in 1.5 mL of pyridine, cooled to ice-bath temperature and 0.3 mL of acetic anhydride was added and the mixture was refrigerated overnight. The solution was diluted with 1 mL of water, stirred for 10 min in the ice bath then distributed between 10 mL of water and 30 mL of ethyl acetate. The organic layer was washed with 4×15 mL of water, once with 5 mL of brine then dried (sodium sulfate) and evaporated. The nearly colorless, oily residue was taken up in 1:6 ethyl acetate-hexane as mobile phase for fractions 1-6 then 1:4 ethyl acetate-hexane was used. Fractions 9-19 (TLC, 1:4 ethyl acetate-hexane, Rf 0.09, see below) were pooled, evaporated, to give 0.0306 g, which was taken up in methyl formate, filtered, then evaporated. It gave 0.0376 of the title compound (18).

### EXAMPLE 9

### Synthesis of 1,3-Di-O-A cetyl-1,25-dihydroxy-16ene-23-yne-19-nor-cholecalciferol (20)

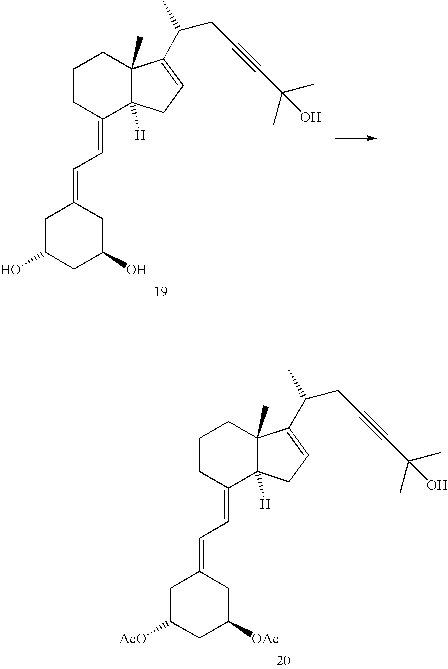

50 mg of 1,25-dihydroxy-16-ene-23-yne-19-nor-cholecalciferol (19) was dissolved in 0.8 mL of pyridine, cooled to ice-bath temperature and 0.2 mL of acetic anhydride was added. The mixture was refrigerated for 3 days then diluted with 1 mL of water, stirred for 10 min in the ice bath and distributed between 5 mL of water and 20 mL of ethyl acetate. The organic layer was washed with 4×5 mL of water, once with 3 mL of brine then dried (sodium sulfate) and evaporated. The nearly colorless, oily residue was taken up in 1:6 ethyl acetate-hexane then flash chromatographed on a 15×120 mm column using 1:6 ethyl acetate-hexane as mobile phase for fractions 1-6, 1:4 for fractions 9-12, 1:3 for fractions 13-15 and 1:2 ethyl acetate-hexane for the remaining fractions. Fractions 11-16 (TLC, 1:4 ethyl acetate-hexane, Rf 0.09, see below) were pooled, evaporated 76.1487−76.1260=0.0227 g, taken up in methyl formate, filtered, then evaporated. It gave 0.0186 g of the title compound (20).

### EXAMPLE 10

### Synthesis of 1,3-Di-O-acetyl-1,25-dihydroxy-16-ene-23-yne-26,2 7-bishomo-19-nor-cholecalciferol (22)

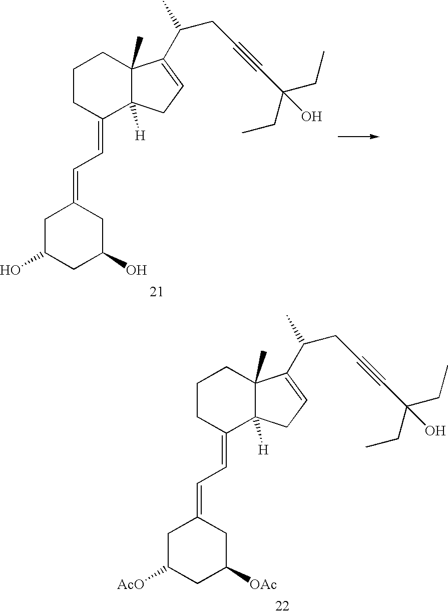

0.0726 g of 1,25-dihydroxy-16-ene-23-yne-26,27-bishomo-I19-nor-cholecalciferol (21) was dissolved in 0.8 mL of pyridine, cooled to ice-bath temperature and 0.2 mL of acetic anhydride was added. The solution was stirred in the ice-bath then refrigerated overnight. The solution was then diluted with 1 mL of water, stirred for 10 min in the ice bath and distributed between 10 mL of water and 25 ML of ethyl acetate. The organic layer was washed with 3×10 mL of water, once with 5 mL of saturated sodium hydrogen carbonate, once with 3 mL of brine then dried and evaporated, 33.5512×33.4654=0.0858 g of a tan oily residue that was flash-chromatographed on a 15×120 mm column using 1:6 as mobile phase. Fractions 7-11 (20 mL each) were pooled (TLC-1:4 ethyl acetate-hexane, Rf 0.14) and evaporated, 67.2834−67.2654=0.018 g. This residue was taken up in methyl formate, filtered and evaporated. It gave 0.0211 g of the title compound (22).

### EXAMPLE 11

### Synthesis of 1,3-Di-O-acetyl-1,25-dihydroxy-20-cyclopropyl-23-yne-19-nor-cholecalciferol (24)

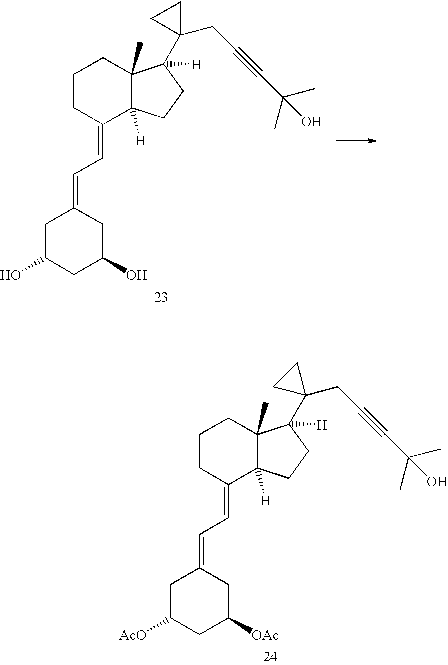

0.282 g of 1,25-Dihydroxy-20-cyclopropyl-23-yne-19-nor-cholecalciferol (23) was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×110 mm column using 1:6 ethyl acetate-hexane as mobile phase for fractions 1-4; 1:4 for fractions 5-12, 1:3 for fractions 13-15 ethyl acetate-hexane for the remaining fractions. Fractions 7-12 (TLC, 1:4 ethyl acetate-hexane, Rf 0.13) were pooled, evaporated, the residue taken up in methyl formate, filtered, then evaporated to give 0.023 g of the title compound (24).

### EXAMPLE 12

### Synthesis of 1,3,25-Tri-O-acetyl-1,25-dihydroxy-20-cyclopropyl-23-yne-26,2 7-hexafluoro-19-nor-cholecalciferol (26) and 1,3-Di-O-acetyl-1,25-dihydroxy-20-cyclopropyl-23-yne-26,2 7-hexafluoro-19-nor-cholecalciferol (27)

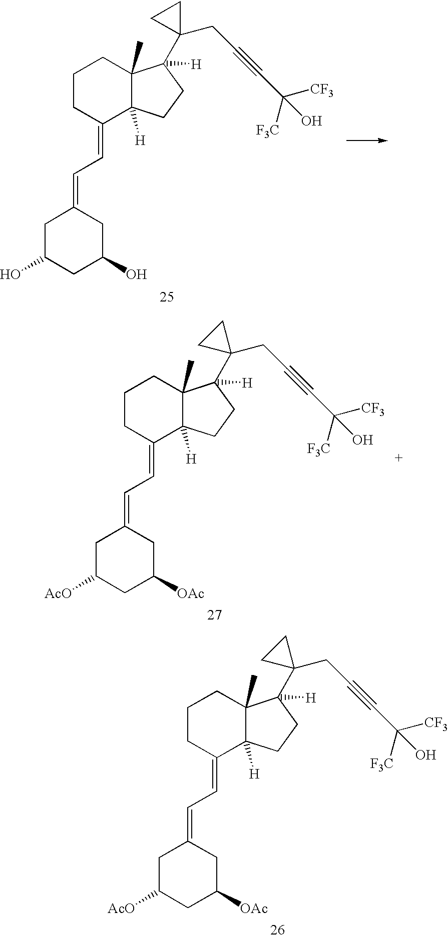

0.1503 g of 1,25-dihydroxy-20-cyclopropyl-23-yne-26,27-hexafluoro-l 9-nor-cholecalciferol (25) was dissolved in 0.8 mL of pyridine, cooled to ice-bath temperature and 0.2 mL of acetic anhydride was added. The mixture was refrigerated overnight then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash chromatographed on a 15×150 mm column using 1:6 ethyl acetate-hexane as mobile phase for fractions 1-5, 1:4 for the remaining fractions. Fractions 3-4 and 6-7 were pooled, evaporated, then taken up in methyl formate, filtered, and evaporated to give 0.0476 g of the title triacetate (26) and 0.04670 g of the title diacetate (27).

### EXAMPLE 13

### Synthesis of 1,3-Di-O-acetyl-1,25-dihydroxy-20-cyclopropyl-23-yne-cholecalciferol (29)

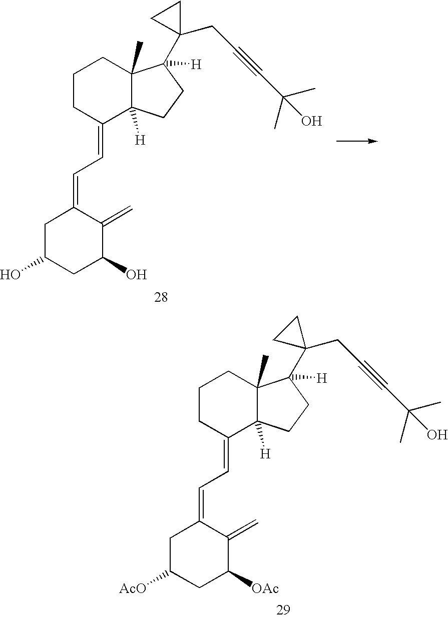

0.0369 g of 1,25-dihydroxy-20-cyclopropyl-23-yne-cholecalciferol (28) was dissolved in 0.8 mL of pyridine, cooled to ice-bath temperature and 0.2 mL of acetic anhydride was added and the mixture was refrigerated overnight, then diluted with 1 mL of water, stirred for 10 min in the ice bath and distributed between 5 mL of water and 20 mL of ethyl acetate. The organic layer was washed with 3×5 mL of water, once with 5 mL of saturated sodium hydrogen carbonate, once with 3 mL of brine then dried (sodium sulfate) and evaporated. The oily residue was taken up in 1:6 ethyl acetate-hexane then flash-chromatographed on a 13×110 mm column using 1:6 ethyl acetate-hexane as mobile phase for fractions 1-7, 1:4 ethyl acetate-hexane for the remaining fractions. Fractions 9-11 (TLC, 1:4 ethyl acetate-hexane) were pooled, evaporated, taken up in methyl formate, filtered, then evaporated, to give 0.0099 g of the title compound (29).

### EXAMPLE 14

### Synthesis of 1,3-Di-O-acetyl-1,25-dihydroxy-20-cyclopropyl-23E-ene-26,27-hexafluoro-19-nor-cholecalciferol (31)

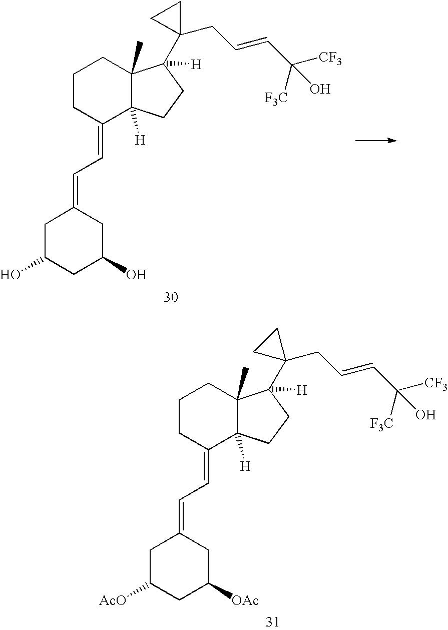

0.0328 g of 1,25-dihydroxy-20-cyclopropyl-23E-ene-26,27-hexafluoro-19-nor-cholecalciferol (30) was dissolved in 0.8 mL of pyridine, cooled to ice-bath temperature and 0.2 mL of acetic anhydride was added. The solution was refrigerated overnight. The solution was then diluted with I mL of water, stirred for 10 min in the ice bath and distributed between 5 mL of water and 20 mL of ethyl acetate. (Extraction of the aqueous layer gave no phosphomolybdic acid-detectable material). The organic layer was washed with 3×5 mL of water, once with 5 mL of saturated sodium hydrogen carbonate, once with 3 mL of brine then dried (sodium sulfate) and evaporated, the residue shows Rf 0.25 as the only spot. The oily residue was taken up in 1:6 ethyl acetate-hexane then flash-chromato-graphed on a 13.5×110 mm column using 1:6 ethyl acetate-hexane as mobile phase for fractions 1-10. Fractions 4-9 were pooled and evaporated, the residue taken up in methyl formate, filtered, then evaporated to give 0.0316 g of the title compound (31).

### EXAMPLE 15

### Synthesis of 1,3-Di-O-acetyl-1,25-dihydroxy-20-cyclopropyl-23Z-ene-26,27-hexafluoro-19-nor-cholecalciferol (33)

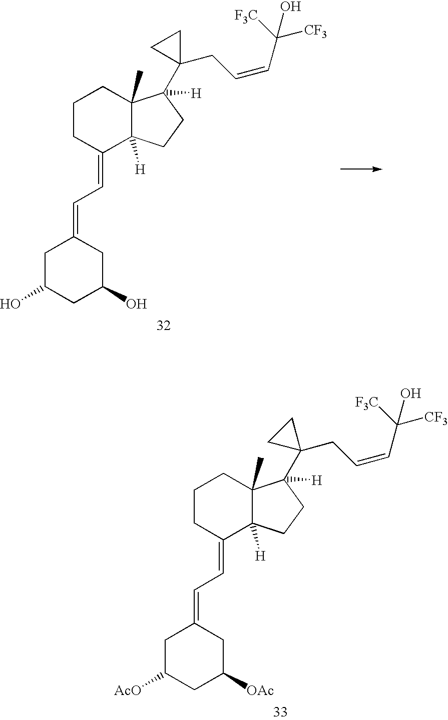

0.0429 g of 1,25-dihydroxy-20-cyclopropyl-23Z-ene-26,27-hexafluoro-19-nor-cholecalciferol (32) was dissolved in 0.8 mL of pyridine, cooled to ice-bath temperature and 0.2 mL of acetic anhydride was added. The solution was refrigerated overnight. The solution-was then diluted with 1 mL of water, stirred for 10 min in the ice bath and distributed between 7 mL of water and 25 mL of ethyl acetate. The organic layer was washed with 3×5 mL of water, once with 5 mL of saturated sodium hydrogen carbonate, once with 3 mL of brine then dried (sodium sulfate, TLC (1:4 ethyl acetate-hexane shows mostly one spot) and evaporated, flash-chromatographed on a 15×120 mm column using 1:6 as mobile phase. Fractions 3-6 (20 mL each) were pooled and evaporated. The residue was taken up in methyl formate, filtered and evaporated, to give 0.0411 g of the title compound (33).

### EXAMPLE 16

### Synthesis of 1,3-Di-O-acetyl-1,25-dihydroxy-20-cyclopropyl-cholecalciferol (35) (“Compound A”)

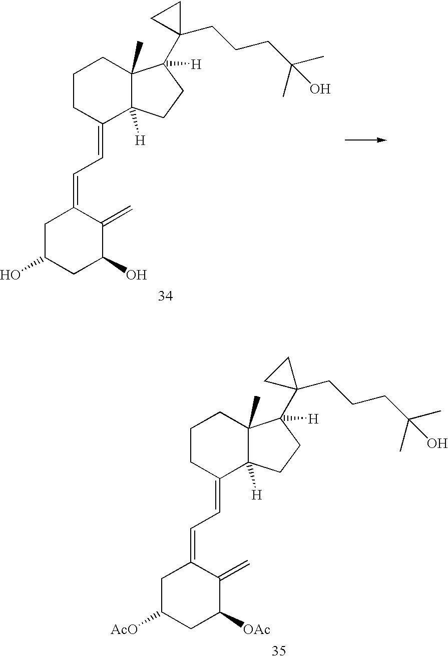

0.0797 g of 1,25-dihydroxy-20-cyclopropyl-cholecalciferol (34) was dissolved in 0.8 mL of pyridine, cooled to ice-bath temperature and 0.2 mL of acetic anhydride was added. The solution was refrigerated overnight. The solution was then diluted with 1 mL of water, stirred for 10 min in the ice bath and distributed between 10 mL of water and 25 mL of ethyl acetate. The organic layer was washed with 3×10 mL of water, once with 5 mL of saturated sodium hydrogen carbonate, once with 3 mL of brine then dried and evaporated, to give 0.1061 g of a tan oily residue that was flash-chromatographed on a 15×120 mm column using 1:6 as mobile phase. Fractions 9-16 (20 mL each) were pooled (TLC 1:4 ethyl acetate-hexane, Rf 0.13) and evaporated. This residue was taken up in methyl formate, filtered and evaporated to give 0.0581 g of the title compound (35).

### EXAMPLE 17

### Synthesis of 1,3-Di-O-acetyl-1α,25-dihydroxy-16-ene-20-cyclopropyl-19-nor-cholecalciferol-(37)

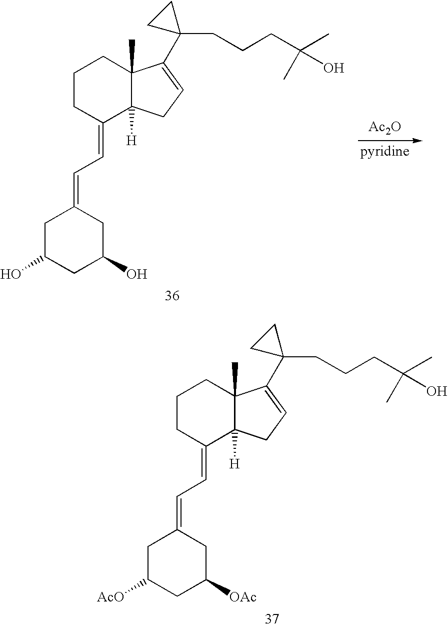

To the solution of 1α,25-Dihydroxy-16-ene-20-cyclopropyl-19-nor-cholecalciferol (36) (94 mg, 0.23 mmol) in pyridine (3 mL) at 0° C., acetic anhydride (0.5 mL, 5.3 mmol) was added. The mixture was stirred for 1 h, refrigerated for 15 h. and then was stirred for additional 8 h. Water (10 mL) was added and after stirring for 15 min. the reaction mixture was extracted with AcOEt: Hexane 1:1 (25 mL), washed with water (4×25 mL) and brine (20 mL), dried over Na₂SO₄. The residue (120 mg) after evaporation of the solvent was purified by FC (15 g, 30% AcOEt in hexane) to give the titled compound (37) (91 mg, 0.18 mmol, 80%). [α]³⁰_{D}=+14.4 c 0.34, EtOH. UV λmax (EtOH): 242 nm (ε 34349), 250 nm (ε 40458), 260 nm (ε 27545); ¹H NMR (CDCl₃): 6.25 (1H, d, J=11.1 Hz), 5.83 (1H, d, J=11.3 Hz), 5.35 (1H, m), 5.09 (2H, m), 2.82-1.98 (7H, m), 2.03 (3H, s), 1.98 (3H, s), 2.00-1.12 (15H, m), 1.18 (6H s), 0.77 (3H, s ),0.80-0.36 (4H, m); ¹³C NMR (CDCl₃): 170.73(0), 170.65(0), 157.27(0), 142.55(0), 130.01(0), 125.06(1), 123.84(1), 115.71(1), 71.32(0), 70.24(1), 69.99(1), 59.68(1), 50.40(0), 44.08(2), 41.40(2), 38.37(2), 35.96(2), 35.80(2), 32.93(2), 29.48(3), 29.31(2), 28.71(2), 23.71(2), 22.50(2), 21.56(3), 21.51(0), 21.44(3), 18.01(3), 12.93(2), 10.53(2); MS HRES Calculated for C₃₁H46O₅ M+Na 521.3237. Observed M+Na 521.3233.

### EXAMPLE 18

### Synthesis of 1,3-Di-O-acetyl-1 a,25-hydroxy-16-ene-20-cyclopropyl-cholecalciferol (39)

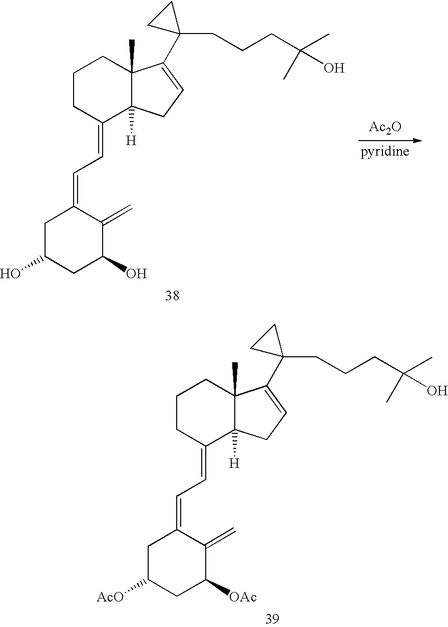

To the solution of 1α,25-Dihydroxy-16-ene-20-cyclopropyl-cholecalciferol (38) (100 mg, 0.23 mmol) in pyridine (3 mL) at 0° C., acetic anhydride (0.5 mL, 5.3 mmol) was added. The mixture was stirred for 2 h and then refrigerated for additional 15 h. Water (10 mL) was added and after stirring for 15 min. the reaction mixture was extracted with AcOEt: Hexane 1:1 (25 mL), washed with water (4×25 mL), brine (20 mL) and dried over Na₂SO₄. The residue (150mg) after evaporation of the solvent was purified by FC (15 g, 30% AcOEt in hexane) to give the titled compound (39) (92 mg, 0.18 mmol, 78%). [α]³⁰_{D}=−14.9 c 0.37, EtOH. UV λmax (EtOH): 208 nm (ε 15949), 265 nm (ε 15745); ¹H NMR (CDCl₃): 6.34 (1H, d, J=11.3 Hz), 5.99 (1H, d, J=11.3 Hz), 5.47 (1H, m), 5.33 (1H, m), 5.31 (1H, s), 5.18 (1H, m), 5.04(1H, s), 2.78 (1H, m), 2.64 (1H, m), 2.40-1.10 (18H, m), 2.05 (3H, s), 2.01 (3H, s), 1.18 (6H, s), 0.76 (3H, s),0.66-0.24 (4H, m); ¹³C NMR (CDCl₃): 170.76(0), 170.22(0), 157.18(0), 143.02(0), 142.40(0), 131.94(0), 125.31(1), 125.10(1), 117.40(1), 115.22(2), 72.97(1), 71.32(0), 69.65(1), 59.71(1), 50.57(0), 44.07(2), 41.73(2), 38.36(2), 37.10(2), 35.80(2), 29.45(3), 29.35(2), 29.25(3), 28.92(2), 23.80(2), 22.48(2),21.55(3),21.50(3), 21.35(0), 17.90(3), 12.92(2), 10.54(2); MS HRES Calculated for C₃₂H₄₆O₅ M+Na 533.3237. Observed M+Na 533.3236.

### EXAMPLE 19

### Synthesis of 1,3-Di-O-acetyl-1,25-dihydroxy-23-yne-cholecalciferol (41)

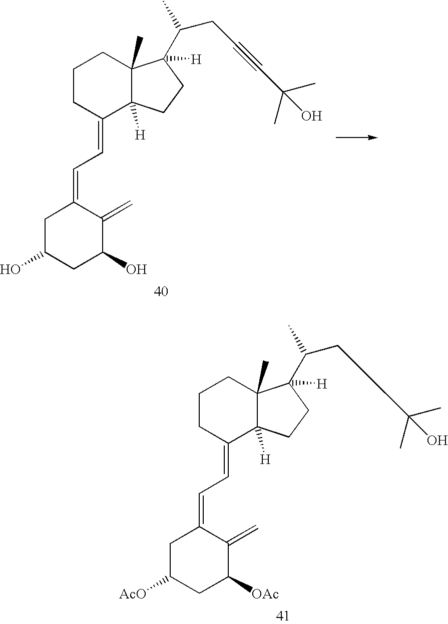

0.2007 g of 40(0.486 mmol) was dissolved in 2 mL of pyridine. This solution was cooled in an ice bath and 0.6 mL of acetic anhydride was added. The solution was kept in an ice bath for 45 h then diluted with 10 mL of water, stirred for 10 min and equilibrated with 10 mL of water and 40 mL of ethyl acetate. The organic layer was washed with 4×20 mL of water, 10 mL of brine, dried (sodium sulfate) and evaporated. The brown, oily residue was flash chromatographed using 1:19, 1:9, and 1:4 ethyl acetate-hexane as stepwise gradient. The main band with Rf 0.16 (TLC 1:4 acetate-hexane) was evaporated to give 1,3-di-O-acetyl-1,25-dihydroxy-23-yne-cholecalciferol (41) a colorless foam, 0.0939 g.

Biological Assays and Data

As described in the following examples, the Inventors' finding that calcitriol and Vitamin D₃ analogues can have an effect on the growth and function of bladder cells has been proven in in vitro models by culturing human stromal bladder cells and has been confirmed in a preclinical in vivo validated model.

### EXAMPLE 20

### Determination of Maximum Tolerated Dose (MTD)

The maximum tolerated dose of the vitamin D₃ compounds of the invention were determined in eight week-old female C57BL/6 mice (3 mice/group) dosed orally (0.1 ml/mouse) with various concentrations of Vitamin D₃ analogs daily for four days. Analogs were formulated in miglyol for a final concentration of 0.01, 0.03, 0.1 0.3, 1, 3, 10, 30, 100 and 300 μg/kg when given at 0.1 ml/mouse p.o. daily. Blood for serum calcium assay was drawn by tail bleed on day five, the final day of the study. Serum calcium levels were determined using a colorimetric assay (Sigma Diagnostics, procedure no. 597). The highest dose of analog tolerated without inducing hypercalcemia (serum calcium>10.7 mg/dl) was taken as the maximum tolerated dose (MTD). Table 3 shows the relative MTD for various vitamin D₃ compounds. Notably, compound (2) has an MTD that is more than 300 times greater than compound (1). Similarly, compounds (4) and (5) also have a MTD that is considerably-greater than their parent compound (3).

### EXAMPLE 21

### Immunological Assay

Immature dendritic cells. (DC) were prepared as described in Romani, N. et al. (Romani, N. et al. (1996) J. Immunol. Meth. 196:137). IFN-γ production by allogeneic T cell activation in the mixed leukocyte response (MLR) was determined as described in Penna, G., et al., J Immunol., 164: 2405-2411 (2000).

Briefly, peripheral blood mononuclear cells (PBMC) were separated from buffy coats by Ficoll gradient and the same number (3×10⁵) of allogeneic PBMC from 2 different donors were co-cultured in 96-well flat-bottom plates. The vitamin D₃ compounds were added to each of the cultures. After 5 days, IFN-γ production in the MLR assay was measured by ELISA and the results expressed as amount (nM) of test compound required to induce 50% inhibition of IFN-γ production (IC₅₀). The results are summarized in Table 3.

TABLE 3

| | MTD (mice) | INF-γ |
|---|---|---|
| Compound | μg/kg | IC₅₀ pM |
| 1,25(OH)₂D₃ | 1 | 22 |
| 1,25-dihydroxy-16,23Z-diene-26,27- | 0.03 | 0.3 |
| hexafluoro-19-nor-cholecalciferol (1) | | |
| 1,3-di-O-acetyl-1,25-dihydroxy- | 0.1 | 722.0 |
| 16,23Z-diene-26,27-hexafluoro-19-nor- | | |
| cholecalciferol (2) | | |
| 1,25-dihydroxy-16-ene-23-yne-26,27- | 0.3 | 1.5 |
| hexafluoro-19-nor-cholecalciferol (3) | | |
| 1,3-Di-O-acetyl-1,25-Dihydroxy-16- | 10 | 525.0 |
| ene-23-yne-26,27-hexafluoro-19-nor- | | |
| cholecalciferol (4) | | |
| 1,3,25-Tri-O-acetyl-1,25-Dihydroxy-16- | 3 | 499.0 |
| ene-23-yne-26,27-hexafluoro-19-nor- | | |
| cholecalciferol (5) | | |
| 1,3-Di-O-acetyl-1,25-dihydroxy-16-ene- | 10 | 51.0 |
| 23-yne-cholecalciferol (7) | | |
| 1,3-Di-O-acetyl-1,25-dihydroxy-16,23E- | 3 | 13.0 |
| diene-cholecalciferol (9) | | |
| 1,3-Di-O-acetyl-1,25-dihydroxy-16-ene- | 1 | 36.0 |
| cholecalciferol (11) | | |
| 1,3,25-Tri-O-acetyl-1,25-dihydroxy-16- | 3 | 40.1 |
| ene-23-yne-26,27-hexafluoro- | | |
| cholecalciferol (13) | | |
| 1,3-Di-O-acetyl-1,25-dihydroxy-16-ene- | 10 | 27.3 |
| 23-yne-26,27-hexafluoro-cholecalciferol (14) | | |
| 1,3-Di-O-acetyl-1,25-dihydroxy-16,23E- | 0.3 | 51.3 |
| diene-25R,26-trifluoro-cholecalciferol (16) | | |
| 1,3-Di-O-acetyl-1,25-dihydroxy-16-ene- | 3 | 3.0 |
| 19-nor-cholecalciferol (18) | | |
| 1,3-Di-O-Acetyl-1,25-dihydroxy-16-ene- | 30 | 25.0 |
| 23-yne-19-nor-cholecalciferol (20) | | |
| 1,3-Di-O-acetyl-1,25-dihydroxy-16-ene- | 100 | 25.3 |
| 23-yne-26,27-bishomo-19-nor- | | |
| cholecalciferol (22) | | |
| 1,3-Di-O-acetyl-1,25-dihydroxy-20- | 100 | 802.0 |
| cyclopropyl-23-yne-19-nor- | | |
| cholecalciferol (24) | | |
| 1,3,25-Tri-O-acetyl-1,25-dihydroxy-20- | 10 | 922.0 |
| cyclopropyl-23-yne-26,27-hexafluoro- | | |
| 19-nor-cholecalciferol (26) | | |
| 1,3-Di-O-acetyl-1,25-dihydroxy-20- | 10 | 78.0 |
| cyclopropyl-23-yne-26,27-hexafluoro- | | |
| 19-nor-cholecalciferol (27) | | |
| 1,3-Di-O-acetyl-1,25-dihydroxy-20- | 30 | 7.8 |
| cyclopropyl-23-yne-cholecalciferol (29) | | |
| 1,3-Di-O-acetyl-1,25-dihydroxy-20- | 0.3 | 0.8 |
| cyclopropyl-23E-ene-26,27-hexafluoro- | | |
| 19-nor-cholecalciferol (31) | | |
| 1,3-Di-O-acetyl-1,25-dihydroxy-20- | 10 | 99.0 |
| cyclopropyl-23Z-ene-26,27-hexafluoro- | | |
| 19-nor-cholecalciferol (33) | | |
| 1,3-Di-O-acetyl-1,25-dihydroxy-20- | 30 | 2.7 |
| cyclopropyl-cholecalciferol (35) | | |
| (“Compound A”) | | |
| 1,3-Di-O-acetyl-1α,25-dihydroxy- | 10 | 68.0 |
| 16-ene-20-cyclopropyl-19-nor- | | |
| cholecalciferol (37) | | |
| 1,3-Di-O-acetyl-1α,25-hydroxy- | 3 | 45.0 |
| 16-ene-20-cyclopropyl- | | |
| cholecalciferol (39) | | |
| 1,3-Di-O-acetyl-1,25-dihydroxy- | 1 | 80.0 |
| 23-yne-cholecalciferol (41) | | |

### EXAMPLE 22

### Capacity of Calcitriol and Vitamin D Analogues to Inhibit Experimental Autoimmune Uveoretinitis(EAU)

As described in the following examples, the Inventors' finding that VDR agonists such as Vitamin D3 analogues can have an effect on uveitis has been proven in an in vivo model.

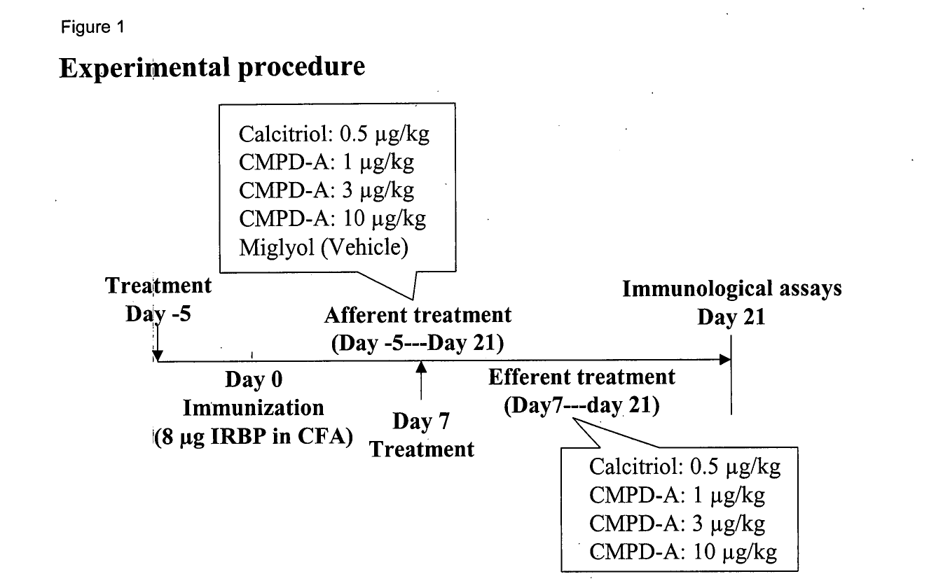

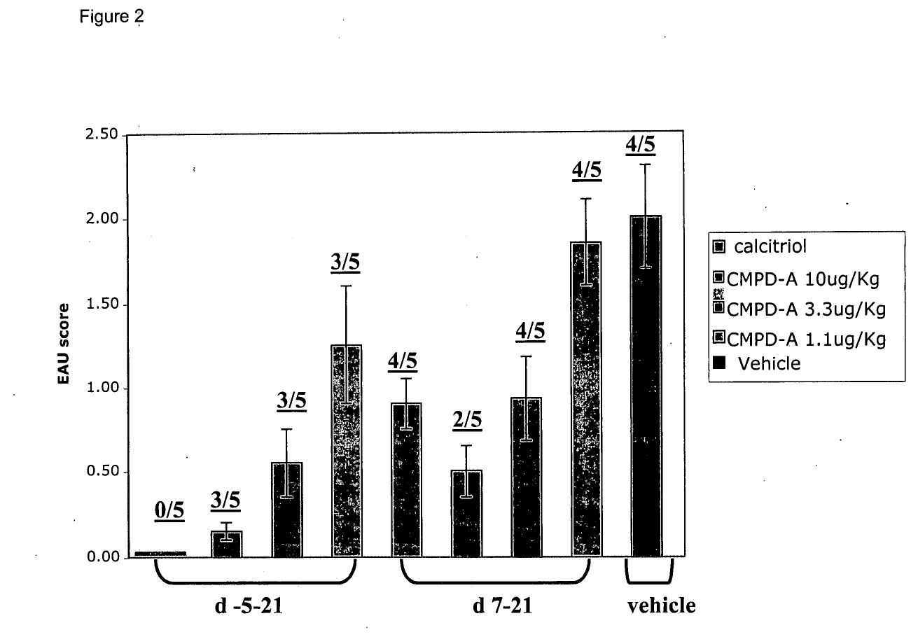

EAU an autoimmune disease mediated by Th1-type uveitogenic CD4+ T cells serves as a model for human posterior uveitis. Following the experimental procedure shown in FIG. 1, EAU-susceptible B10.RIII mice were immunized with an uveitogenic regimen of 8 μg of interphotoreceptor retinoid-binding protein (IRBP) in CFA and treated orally with calcitriol or with Compound A (1,3-Di-O-acetyl-1,25-dihydroxy-20-cyclopropyl-cholecalciferol), before or after EAU induction. EAU development was followed by funduscopy and confirmed by histopathology on eyes collected 21 days after immunization. FIG. 2 shows the EAU disease score (quantitated between 0 and 4) at day 21.

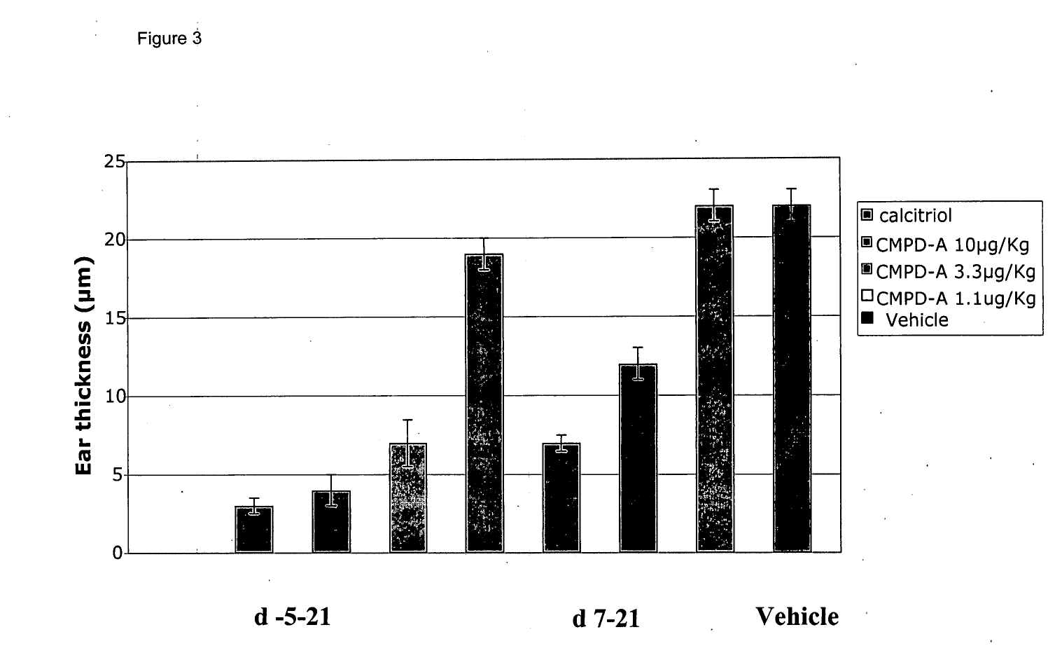

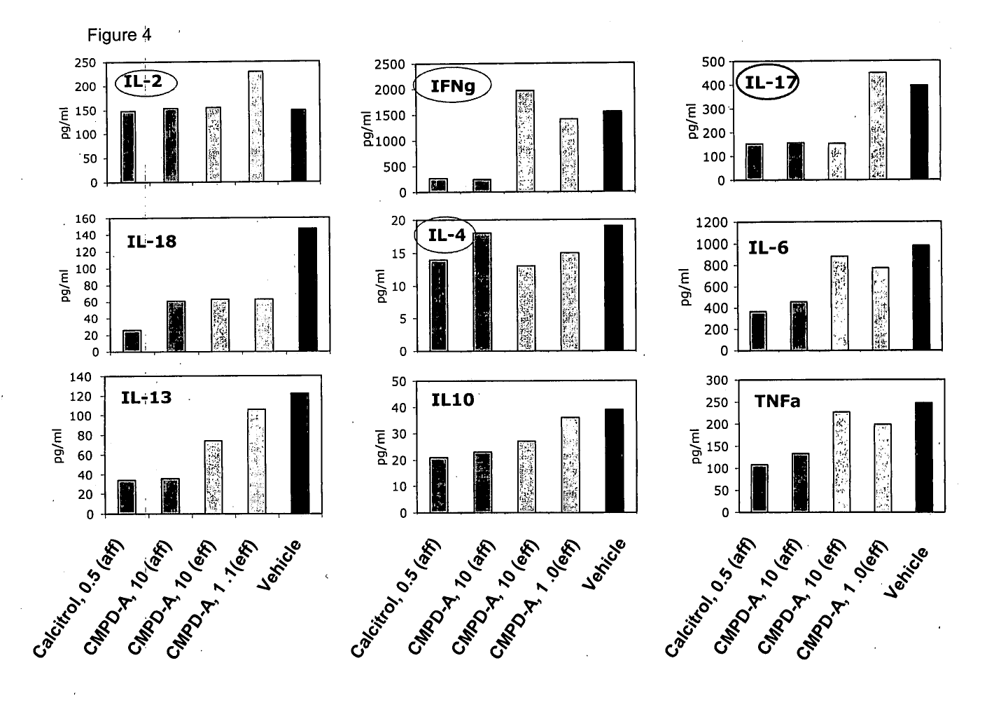

The Inventors found that calcitriol at 0.5 μg/kg and Compound A at 10 μg/kg can prevent EAU, when administered from day −6 to 2. In addition, Compound A but not calcitriol, could inhibit EAU development when treatment was started 7 days after immunization. As shown in FIG. 3, protected mice had reduced antigen-specific delayed type hypersensitivity (DTH) responses to IRBP. It can be concluded that delayed hypersensitivity to IRBP in treated mice is correlated with disease severity. As shown in FIG. 4, in vitro assay of primed lymph node cells (LN) indicated that calcitriol and Compound A both appeared to be potent not on the inhibition of specific T cell proliferation, but also on T cell polarization. Preventive treatment remarkably diminished both IFN-gamma and IL-17 production by LN. Both of these cytokines have been found to be produced by pathogenic T-cells in TL1-type autoimmune diseases.

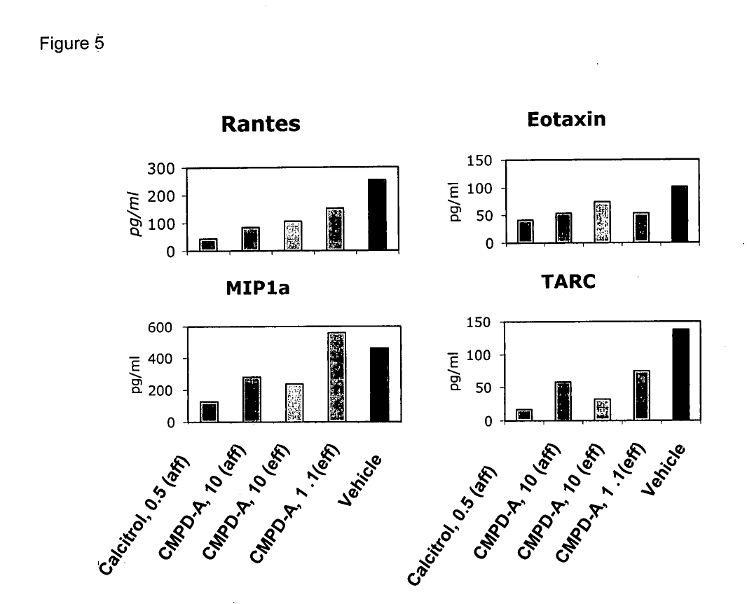

Therapeutic administration with Compound A markedly inhibited IL-17 but not IFN-g production. While combined data indicated that IL-17 releasing profile was more consistent to the histology measurement rather than IFN-g. In consistency with disease amelioration, Ag driven chemokine release, such as MIP-1a, Rantes and TARC, is inhibited by Vitamin D3 derivatives as shown in FIG. 5. The further mechanism(s) involved in inhibition of EAU by VDR agonists are being investigated. In conclusion, the inventors have demonstrated that natural VitD3 (calcitriol) effectively prevents EAU whilst a synthetic Vitamin D receptor (VDR) agonist, Compound A, is capable of preventing as well as treating EAU.

### EXAMPLE 23

### Soft Gelatin Capsule Formulation I

| Item | Ingredients | mg/Capsule |
|---|---|---|
| 1 | Compound (35) from Example 16 | 10.001-0.02 |
| 2 | Butylated Hydroxytoluene (BHT) | 0.016 |
| 3 | Butylated Hydroxyanisole (BHA) | 0.016 |
| 4 | Miglyol 812 qs. | 160.0 |

Manufacturing Procedure:

1. BHT and BHA is suspended in Miglyol 812 and warmed to about 50° C. with stirring, until dissolved.

2. 1,3-Di-O-acetyl-1,25-dihydroxy-20-cyclopropyl-cholecalciferol (35) is dissolved in the solution from step 1 at 50° C.

3. The solution from Step 2 is cooled at room temperature.

4. The solution from Step 3 is filled into soft gelatin capsules. Note: All manufacturing steps are performed under a nitrogen atmosphere and protected from light.

### EXAMPLE 28

### Soft Gelatin Capsule Formulation II

| Item | Ingredients | mg/Capsule |
|---|---|---|
| 1 | Compound (35) from Example 1 | 10.001-0.02 |
| 2 | di-.alpha.-Tocopherol | 0.016 |
| 3 | Miglyol 812 qs. | 160.0 |

Manufacturing Procedure:

1. Di-α-Tocopherol is suspended in Miglyol 812 and warmed to about 50° C. with stirring, until dissolved.

2. 1,3-Di-O-acetyl-1,25-dihydroxy-20-cyclopropyl-cholecalciferol (35) is dissolved in the solution from step 1 at 50° C.

3. The solution from Step 2 is cooled at room temperature.

4. The solution from Step 3 is filled into soft gelatin capsules.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method for preventing or treating uveitis in a subject, comprising administering to a subject in need thereof an effective amount of a vitamin D₃ compound of formula I: 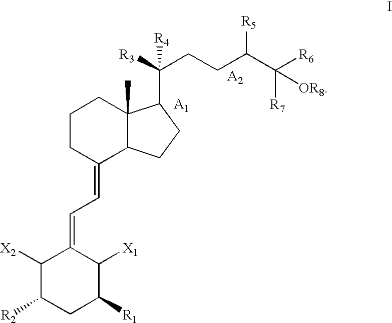
wherein:
A₁ is single or double bond;
A₂ is a single, double or triple bond;
X₁ and X₂ are each independently H₂ or ═CH₂, provided X₁ and X₂ are not both ═CH₂;
R₁ and R₂ are each independently OC(O)C₁-C₄ alkyl, OC(O)hydroxyalkyl, or OC(O)haloalkyl;
R₃, R₄ and R₅ are each independently hydrogen, C₁-C₄ alkyl, hydroxyalkyl, or haloalkyl, with the understanding that R₅ is absent when A₂ is a triple bond, or R₃ and R₄ taken together with C₂₀ form C₃-C₆ cycloalkyl;
R₆ and R₇ are each independently alkyl or haloalkyl; and
R₈ is H, C(O)C₁-C₄ alkyl, C(O)hydroxyalkyl, or C(O)haloalkyl;
and pharmaceutically acceptable esters, salts, and prodrugs thereof such that uveitis is prevented or treated in said subject.

2. The method of claim 1, wherein the vitamin D₃ compound has formula I provided that when A₁ is single bond, R₃ is hydrogen and R₄ is methyl, then A₂ is a double or triple bond.

3. The compound of claim 1, wherein X₁ is H₂ and X₂ is ═CH₂.

4. The compound of claim 1, wherein X₁ and X₂ are H₂.

5. The compound of claim 1, wherein R₄ is C₁-C₄ alkyl.

6. The compound of claim 1, wherein R₃ and R₄, taken together with C₂₀, form C₃-C₆ cycloalkyl.

7. The compound of any preceding claim, wherein R₆ and R₇ are each independently alkyl or haloalkyl.

8. The method of claim 1 having formula I-a

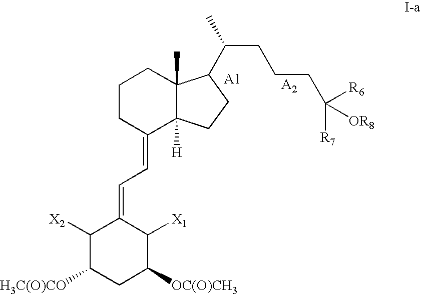

9. The method of claim 8, wherein said compound is 1,3-Di-O-acetyl-1,25-Dihydroxy-16-ene-23-yne-26,27-hexafluoro-19-nor-cholecalciferol; 1,3,25-Tri-O-acetyl-1,25-Dihydroxy-16-ene-23-yne-26,27-hexafluoro-19-nor-cholecalciferol; 1,3-Di-O-acetyl-1,25-dihydroxy-16-ene-19-nor-cholecalciferol; 1,3-Di-O-acetyl-1,25-dihydroxy-16-ene-23-yne-19-nor-cholecalciferol; 1,3-Di-O-acetyl-1,25-dihydroxy-16,23Z-diene-26,27-hexafluoro-19-nor-cholecalciferol; 1,3-Di-O-acetyl-1,25-dihydroxy-16-ene-23-yne-26,27-bishomo-19-nor-cholecalciferol; 1,3-Di-O-acetyl-1,25-dihydroxy-16-ene-23-yne-cholecalciferol; 1,3-Di-O-acetyl-1,25-dihydroxy-16,23E-diene-cholecalciferol; 1,3-Di-O-acetyl-1,25-dihydroxy-16-ene-cholecalciferol; 1,3-Di-O-acetyl-1,25-dihydroxy-16-ene-23-yne-26,27-hexafluoro-cholecalcifero; 1,3-Di-O-acetyl-1,25-dihydroxy-16,23E-diene-25R-26-trifluoro-cholecalciferol; 1,3,25-Tri-O-acetyl-1,25-dihydroxy-16-ene-23-yne-26,27-hexafluoro-cholecalciferol; or 1,3-Di-O-acetyl-1,25-dihydroxy-23-yne-cholecalciferol.

10. The method of claim 1 having formula I-b

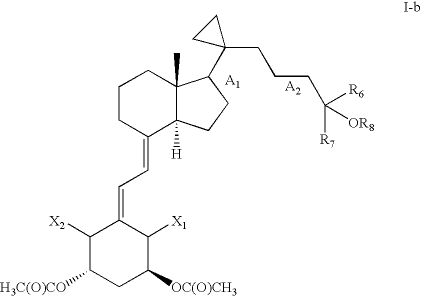

11. The method of claim 10, wherein said compound is 1,3-Di-O-acetyl-1,25-dihydroxy-20-cyclopropyl-23-yne-19-nor-cholecalciferol; 1,3,25-Tri-O-acetyl-1,25-dihydroxy-20-cyclopropyl-23-yne-26,27-hexafluoro-19-nor-cholecalciferol; 1,3-Di-O-acetyl-1,25-dihydroxy-20-cyclopropyl-23-yne-26,27-hexafluoro-19-nor-cholecalciferol; 1,3-Di-O-acetyl-1,25-dihydroxy-20-cyclopropyl-23-yne-cholecalciferol; 1,3-Di-O-acetyl-1,25-dihydroxy-20-cyclopropyl-cholecalciferol; 1,3-Di-O-acetyl-1,25-dihydroxy-20-cyclopropyl-23E-ene-26,27-hexafluoro-19-nor-cholecalciferol; 1,3-Di-O-acetyl-1,25-dihydroxy-20-cyclopropyl-23Z-ene-26,27-hexafluoro-19-nor-cholecalciferol; 1,3-Di-O-acetyl-1,25-dihydroxy-16-ene-20-cyclopropyl-19-nor-cholecalciferol; or 1,3-Di-O-acetyl-1,25-dihydroxy-16-ene-20-cyclopropyl-cholecalciferol.

12. The method of claim 11, wherein said compound is 1,3-Di-O-acetyl-1,25-dihydroxy-20-cyclopropyl-cholecalciferol.

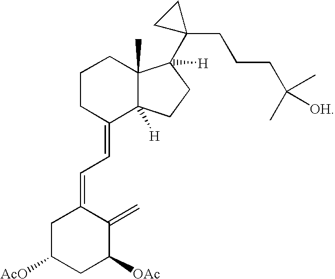

13. The method of claim 1, further comprising identifying a subject in need of prevention or treatment for uveitis.

14. The method of claim 1, further comprising the step of obtaining the vitamin D₃ compound.

15. The method of claim 1, wherein the subject is a mammal.

16. The method of claim 1, wherein the subject is a human.

17. The method of claim 1, wherein the vitamin D₃ compound is formulated in a pharmaceutical composition together with a pharmaceutically acceptable diluent or carrier.

18. The method of claim 1, wherein the vitamin D₃ compound is administered systemically.

19. A packaged formulation for use in the treatment of uveitis, comprising a pharmaceutical composition comprising a compound recited in claim 1 and instructions for use in the treatment of uveitis.

20. A pharmaceutical formulation comprising a therapeutically effective amount for prevention and/or treatment of uveitis of a compound as recited in claim 1 and a pharmaceutically acceptable carrier.
